(19) United States
(12) Patent Application Publication
(10) Pub. No.: US 2004/0243133 A1
(43) Pub. Date: Dec. 2, 2004

(54) Method and system for manufacturing biomedical articles, such as using biomedically compatible infiltrant metal alloys in porous matrices

(75) Inventor: Peter A. Materna, Metuchen, NJ (US)

Correspondence Address: SEED INTELLECTUAL PROPERTY LAW GROUP PLLC, 701 FIFTH AVE, SUITE 6300, SEATTLE, WA, 98104-7092, US

(73) Assignee: Therics, Inc., Princeton, NJ

(21) Appl. No.: 10/794,802

(22) Filed: Mar. 5, 2004

Related U.S. Application Data
(60) Provisional application No. 60/452,795, filed on Mar. 5, 2003.

(51) Int. Cl. A61F 2/28; C22C 14/00

(52) U.S. Cl. 606/76; 420/417; 420/422; 420/425; 623/23.55; 623/23.5

(57) ABSTRACT

Various elements and alloys selected to achieve both biocompatibility and low melting point for use in infiltrating a porous matrix. The infiltrated porous matrix may be made of ceramic, metal, bioglass, or other suitable material. The infiltrated matrix may be used as a biomedical implant, such as for bone repair and regeneration. The matrix may be manufactured using solid free form fabrication techniques such as three-dimensional printing.

### BACKGROUND OF THE INVENTION

1. Field of the Invention

The present invention is directed to biomedically relevant metals and to methods and systems for using biomedically compatible infiltrant metal alloys such as in porous matrix implants.

2. Description of the Related Art

Solid free form fabrication (SFF) has allowed for the manufacture of articles of great geometric complexity quickly and without the use of tooling. Selective laser sintering, stereolithography and three-dimensional printing are all methods of SFF. Articles obtained directly from the three-dimensional printing process have usually been porous, but for many applications a solid article would be preferable. The options for achieving a solid article have basically been either sintering the article to collapse the pores or infiltrating the article with an infiltrant to fill the pores. With infiltration, the infiltrant has had to have a lower melting point than the melting point or thermal damage point of the article being infiltrated.

In regard to infiltrants that are metals, there have been several reports of infusing a relatively low melting point metal into a porous article made from metal powder. A thesis by Ram Chilukuri (Design and Fabrication of a Post-Processing Furnace for 3D Printed Parts, M.S. thesis by Ram Chilukuri, MIT, May 2000) reports infusing copper or copper alloys into stainless steel powder.

A thesis by Diana Buttz (Materials Systems for Low Shrinkage Metal Skeletons in Three-dimensional Printing, M.S. thesis by Diana Buttz, MIT, May 2001) reports infusing nickel-based commercial brazing alloys, which contain nickel along with one or more of silicon or boron or phosphorus as melting point depressants, into molybdenum or tungsten or tantalum powder.

Work by Adam Lorenz et al. (Homogeneous Metal Parts by Infiltration, by Adam Lorenz, Ely Sachs, Sam Allen and Michael Cima, Proceedings of the Symposium on Solid Freeform Fabrication, 2001, pp. 69-76) also used an infiltrant which was a nickel silicon alloy, but the powder into which the infiltrant was infused was pure nickel. This material combination exhibited a further phenomenon called Transient Liquid Phase Sintering, with the result that the final product was an essentially homogeneous alloy of nickel and silicon.

Work by Hong,Sachs et al. (Corrosion behavior of advanced titanium-based alloys made by three-dimensional printing (3DP) for biomedical applications, by S.-B. Hong, N. Eliaz, E. M. Sachs, S. M. Allen, R. M. Latanision, Corrosion Science 43 (2001) 1781-1791, and similar article M. Res. Soc. Symp. Proc. Vol. 662 (2001)), directed toward an implant, used a porous matrix which was a titanium-silver alloy and infiltrated it with tin. However, this work reports that the article infused with tin exhibited a deteriorated corrosion resistance, and that the composition was not considered a promising approach for fabrication of titanium-based implantable prostheses.

A problem with all of these infiltrant metals has been that they have not been suitable for use inside the human body. Either the infiltrant has been subject to corrosion or has been toxic, or the melting point depressant additive has been undesirable in the body. No metal alloy has yet been identified that has had a sufficiently low melting point to be useful as an infiltrant, such as a melting point of less than 1200° C., and also has been biocompatible for use inside the human body.

Caldarese has worked on medical applications involving casting metals into ceramic molds and, in U.S. Pat. No. 5,716,414, has reported an implantable prosthesis which contains a ceramic and also a metal cast into the ceramic. However, beyond citing the general material categories of ceramic and metal, Caldarese has given no example substances of either the ceramic or the metal, or requirements as to melting point of the metal, or properties of the ceramic. Also, U.S. Pat. No. 5,716,414 only discloses casting, which requires providing cavities, sprues and runners at a somewhat macroscopic size scale, as opposed to infiltrating within pores of a porous matrix.

In biomedical applications which do not involve the use of multiple materials within an article, various metals and alloys have become accepted for use inside the body. Titanium and titanium-based alloys have been used in fracture repair, in joint replacement and in endosseous implants for teeth. Alloys combining Ti and Zr have been formulated so as to achieve a desired elastic modulus, as in U.S. Pat. No. 5,169,597 to Davidson. Zirconium and zirconium alloys have been used such as in knee replacement. Niobium has also been considered biocompatible. There has been biomedical use, such as for stents, of nickel-titanium alloys of the very specific composition (from 54 wt % Ni, 46 wt % Ti, to 56 wt % Ni, 44 wt % Ti) which provides shape memory and super elasticity properties.

Many nickel alloys, including the stainless steel family, have been found to be suitable for either implantable or temporary use in medical applications. All of these metals and alloys just described, however, have had melting points that are too high to be useful as infiltrants in situations of practical interest for medical purposes. The elements referred to above have fairly high melting points and the alloys referred to above have not had sufficiently low melting points.

For example, the melting point of nickel-titanium alloys that exhibit shape memory effect is approximately 1300° C. Known binary phase diagrams are given in FIGS. 1, 2 and 3 for nickel-titanium, nickel-zirconium and nickel-niobium, respectively (taken from American Society for Metals Handbook, Volume 3).

Porous ceramic implants such as for bone repair and regeneration have come into use. The low mechanical strength of such implants, however, has limited their use to lightly loaded body parts or to situations in which other structural support exists. Thus, when initially implanted, these porous ceramic implants are not load bearing. This has been a significant limitation on the use of porous ceramics for bone repair and regeneration.

### BRIEF SUMMARY OF THE INVENTION

The invention includes alloys containing metals that are generally known to be acceptable for use in and around the human body such that the alloys have melting points low enough to be useful as infiltrants. The alloys include nickel-titanium alloys; nickel-zirconium alloys; nickel-niobium alloys; alloys resembling stainless steel combined with titanium and/or zirconium and/or niobium; and still other alloys.

The alloys can be used to infiltrate matrices that are either porous metal or porous ceramic. The alloys can also be used to fill cavities or molds by casting. The invention also includes methods of manufacturing biomedical articles containing metal infiltrants, and articles so manufactured. The manufacturing method can include three-dimensional printing. If the matrix is a metal, the invention may include the process of Transient Liquid Phase Sintering.

If the matrix is a porous ceramic which is resorbable, such as tricalcium phosphate, and if the infiltrant is a metal, it is possible to produce an implant which is strong enough to carry at least some load at time of implantion, and which can eventually become completely integrated with bone as the resorbable ceramic degrades and the metal network becomes integrated with natural bone.

In accordance with aspects of the present invention, a biocompatible metal alloy whose melting point is as low as possible is infiltrated into a porous matrix. In one embodiment, the biocompatible metal infiltrant has a melting point below approximately 80% of the melting point of the matrix material (on an absolute temperature scale) or less than a thermal damage temperature. For example, for infiltrating a porous matrix of titanium, a biocompatible metal infiltrant whose melting point is below approximately 1300° C. is provided. For infiltrating a stainless steel matrix, a biocompatible metal infiltrant whose melting point is below approximately 1200° C. is provided.

Bone-like ceramics of the calcium phosphate family require a sintering or processing temperature which is not more than approximately 1200° C. to 1300° C. Accordingly, for infiltrating a matrix comprising ceramics of the calcium phosphate family, a biocompatible metal infiltrant whose melting point is below approximately 1200° C. is provided. For all of these applications, a metal infiltrant with a melting point below 1200° C., and more desirably below 1100° C., and still more desirably below 1000° C. is disclosed in accordance with aspects of the present invention. The constituents of the infiltrant may be titanium, nickel, zirconium, niobium and the major constituents of stainless steel in relative proportions approximating their proportions in stainless steel.

If both the matrix material and the infiltrant are metals, the combination of matrix metal and infiltrant may be suitable for achieving Transient Liquid Phase Sintering. If the matrix is a ceramic, the matrix may be a resorbable ceramic such as tricalcium phosphate.

### BRIEF DESCRIPTION OF THE SEVERAL VIEWS OF THE DRAWINGS

FIG. 1 illustrates a known binary phase diagram for nickel and titanium in accordance with the prior art.

FIG. 2 illustrates a known binary phase diagram for nickel and zirconium in accordance with the prior art.

FIG. 3 illustrates a known binary phase diagram for nickel and niobium in accordance with the prior art.

FIG. 4 illustrates a binary phase diagram for nickel and titanium with compositions of the present invention highlighted in accordance with principles of the present invention.

FIG. 5 illustrates a binary phase diagram for nickel and zirconium with compositions of the present invention highlighted in accordance with principles of the present invention.

FIG. 6 illustrates a binary phase diagram for nickel and niobium with compositions of the present invention highlighted in accordance with principles of the present invention.

FIG. 7 illustrates a pseudo binary phase diagram for stainless steel and titanium with compositions of the present invention highlighted in accordance with principles of the present invention.

FIG. 8 illustrates a pseudo binary phase diagram for stainless steel and zirconium with compositions of the present invention highlighted in accordance with principles of the present invention.

FIG. 9 illustrates a pseudo binary phase diagram for stainless steel and niobium with compositions of the present invention highlighted in accordance with principles of the present invention.

FIGS. 10A-10G illustrates a various ternary alloy phase diagrams for compositions of the present invention in accordance with principles of the present invention.

FIG. 11 illustrates an article having a matrix that is ceramic and an infiltrant that is of a metal composition in accordance with principles of the present invention.

FIGS. 12A-12C illustrate an article having a metal matrix and an infiltrant that is of a liquid metal composition in accordance with principles of the present invention.

FIG. 13 illustrates an article having a matrix that is ceramic and has been partially removed, and an infiltrant that is of a metal composition in accordance with principles of the present invention.

FIG. 14 illustrates an infiltrated matrix having a channel therein in accordance with principles of the present invention.

FIG. 15 illustrates an infiltrated matrix having a partially removed ceramic matrix in accordance with principles of the present invention.

FIG. 16 illustrates a partially infiltrated matrix in accordance with principles of the present invention.

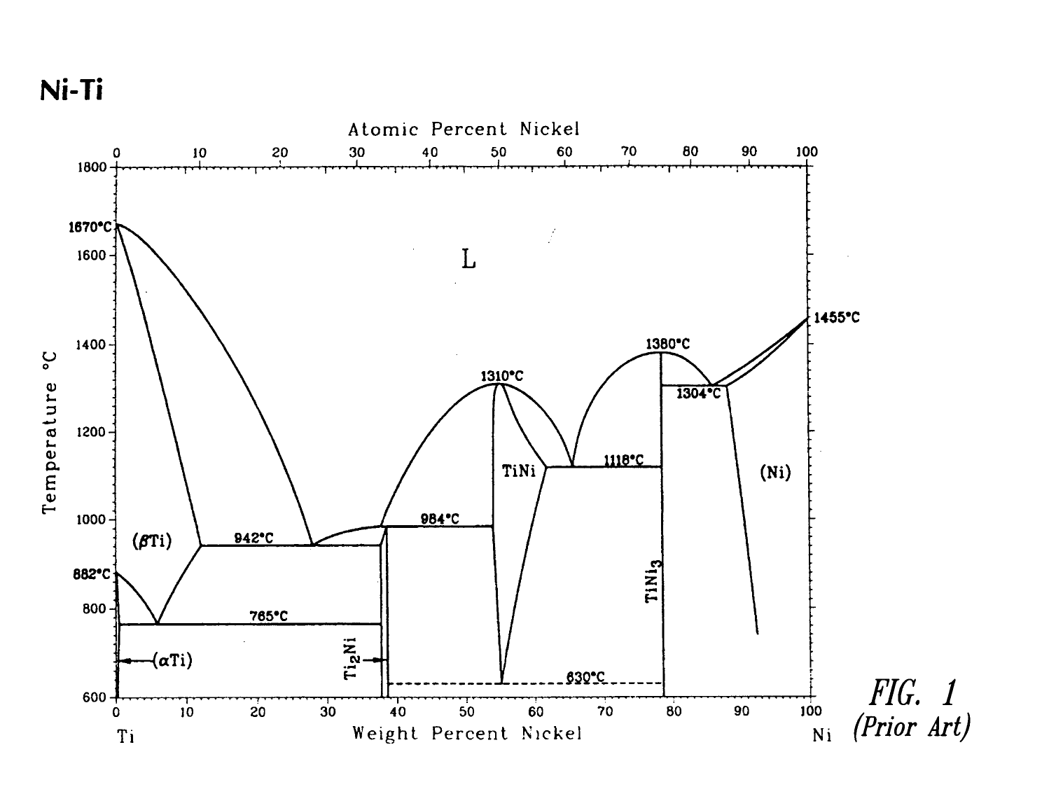

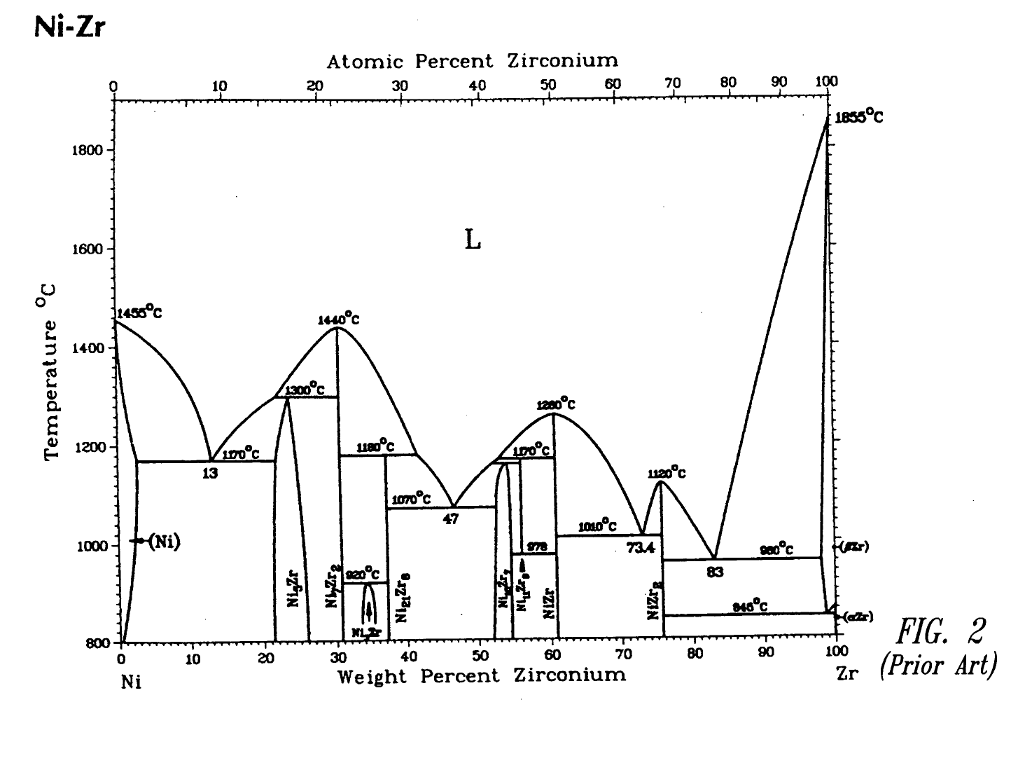

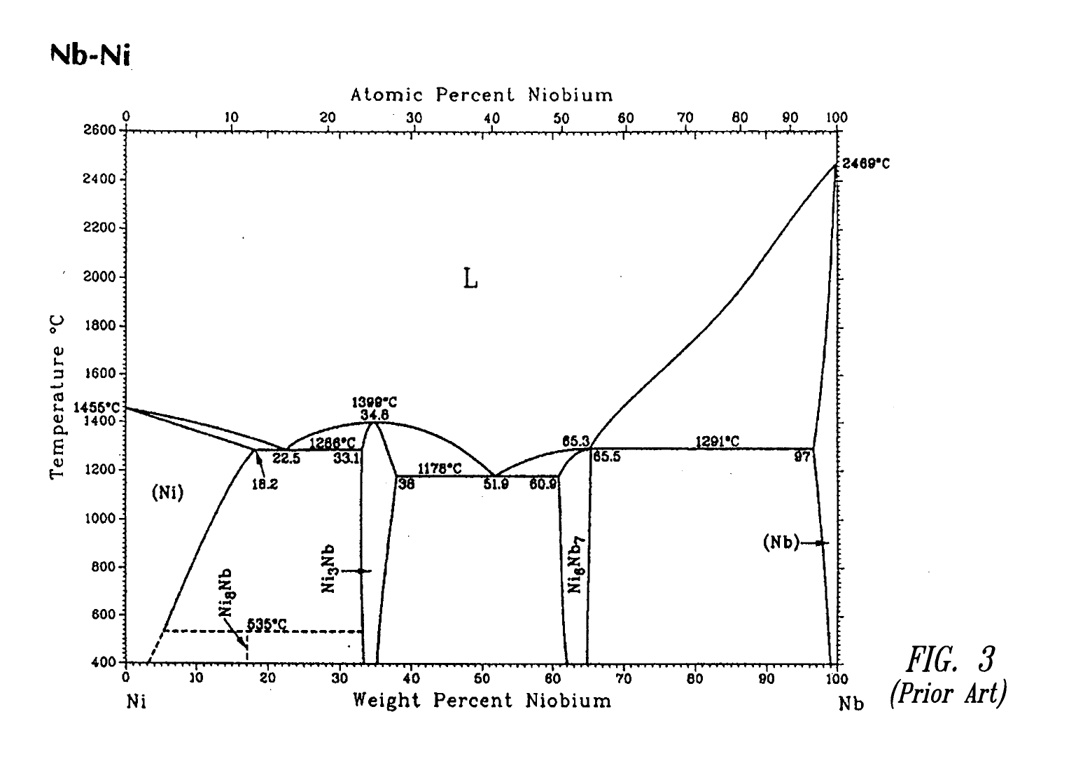

### DETAILED DESCRIPTION OF THE INVENTION

The invention includes the use of various elements and alloys selected to achieve both biocompatibility and low melting point for use in infiltrating a porous matrix. The infiltrated porous matrix may be made of ceramic, metal, bioglass, or other suitable material. The infiltrated matrix may be used as an biomedical implant, such as for bone repair and regeneration.

Although it is sometimes possible for subtle details of composition to make the difference between acceptability and non-acceptability of a material for biomedical applications, there are some basic guidelines that underlie the present invention. Some of the alloys of the present invention contain titanium, because titanium is almost universally acceptable for medical applications, both in substantially pure form and as additives to other metals. Some of the alloys of the present invention contain zirconium for the same reason. Some of the alloys of the present invention contain niobium for the same reason. Some of the alloys of the present invention contain nickel because nickel in the form of many different alloys is well tolerated by the body, even though substantially pure nickel is not well tolerated. Alloys of the present invention may comprise the group of metals iron, nickel and chromium in roughly the relative proportions in which they exist in stainless steels, together with at least one other alloying element.

Alloys of the present invention may be free from copper, in any substantial concentration. Alloys of the present invention may be chosen to be substantially free of aluminum, tin, cobalt, chromium, molybdenum, vanadium and manganese, because these are potentially toxic or corrodible elements, but the alloys of the present invention do not absolutely have to be free of these elements because it is known that there are some biocompatible metal alloys which do contain these elements. As far as metalloid elements, boron, silicon, phosphorus and sulfur, which are sometimes used as additives to metals, alloys of the present invention may be substantially free of these elements, although again in very specific cases it may be possible that these elements be included.

As used herein, the term melting point refers to the temperature at which all constituents of an alloy become liquid. In complicated phase diagrams the term melting point refers to the liquidus, i.e., the upper boundary of the solid-liquid two-phase (slushy) region.

The term eutectic refers to a composition which produces a locally lowest melting point as a function of composition. At a eutectic composition, the phase change may go through a direct solid-to-liquid phase transition, avoiding the existence of a two-phase (slushy) region which might occur at other compositions. As reference information, pure titanium has a melting point of 1670° C., pure zirconium has a melting point of 1855° C., pure niobium has a melting point of 2469° C., pure nickel has a melting point of 1455° C., and stainless steels have an effective melting point of 1400 to 1450° C. depending on composition.

Compositions of the present invention may have a melting point less than approximately 1200° C. Furthermore, compositions of the present invention may have an even lower melting point such as less than approximately 1100° C., or even less than approximately 1000° C., or even as low as 942° C.

The invention includes various different alloy compositions and families which are described individually herein.

Compositions Containing Nickel and Titanium

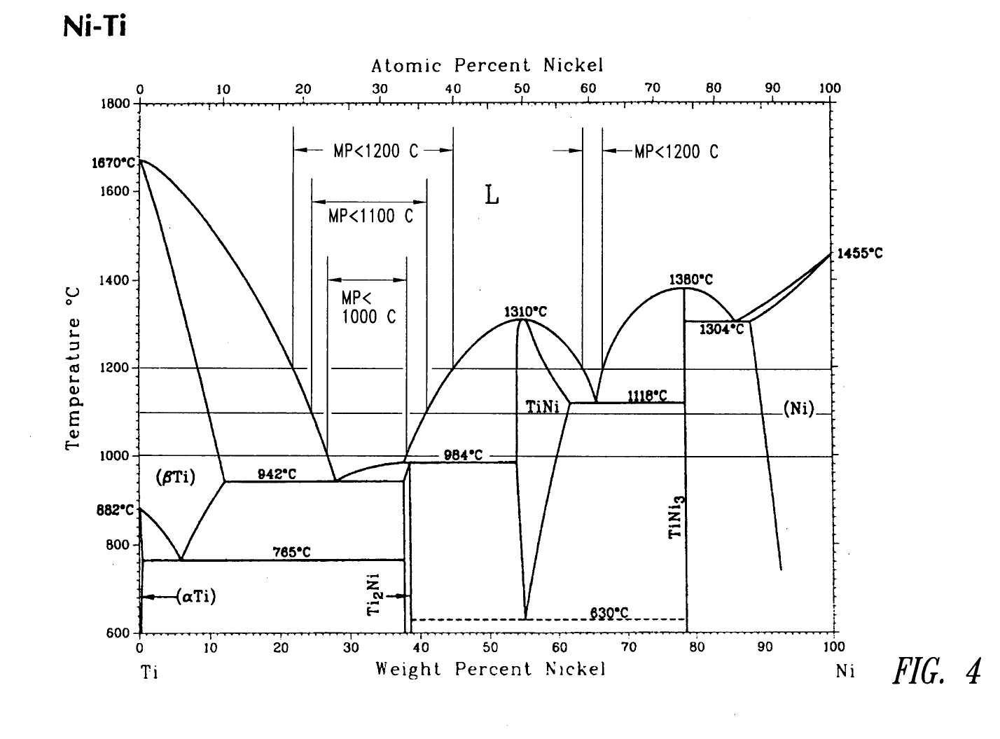

The binary phase diagram of nickel and titanium, illustrating compositions of the present invention, is shown in FIG. 4. The phase diagram has a liquid-region boundary that contains four inflection points or minima.

These points are:

942° C., at 28 wt % Nickel, 72 wt % Titanium;

984° C., at 38 wt % Nickel, 62 wt % Titanium;

1118° C., at 65.5 wt % Nickel, 34.5 wt % Titanium; and

1304° C., at 86 wt % Nickel, 14 wt % Titanium.

The low-melting-point regions near the first two listed points substantially merge with each other. As a result, in the vicinity of the first two listed points, there is a composition range having a melting point of less than 1000° C., which extends from 27 wt % nickel (73 wt % titanium) to 38 wt % nickel (62 wt % titanium). There is a composition range having a melting point of less than 1100° C., which extends from 24.5 wt % nickel (75.5 wt % titanium) to 41 wt % nickel(59 wt % titanium). There is a composition range having a melting point of less than 1200° C., which extends from 22 wt % nickel (78 wt % titanium) to 45 wt % nickel (55 wt % titanium).

In the vicinity of the third listed point there is a composition range having a melting point of less than 1200° C., which extends from 63.5 wt % nickel (36.5 wt % titanium) to 67 wt % nickel (33 wt % titanium).

If it is adequate to have a melting point which is merely less than 1300° C., then the composition can be in the range from 81 wt % Ti (19 wt % Ni) to 48 wt % Ti (52 wt % Ni) or from 43 wt % Ti (57 wt % Ni) to 30 wt % Ti (70 wt % Ni).

The composition of the present invention may contain nickel and titanium in any of the ranges of relative proportions just listed. In addition to the use of alloys containing only nickel and titanium in the stated proportions, it is also possible to use alloys comprising nickel and titanium in the relative proportions as just described, together with a quantity of another constituent(s).

Compositions Containing Nickel and Zirconium

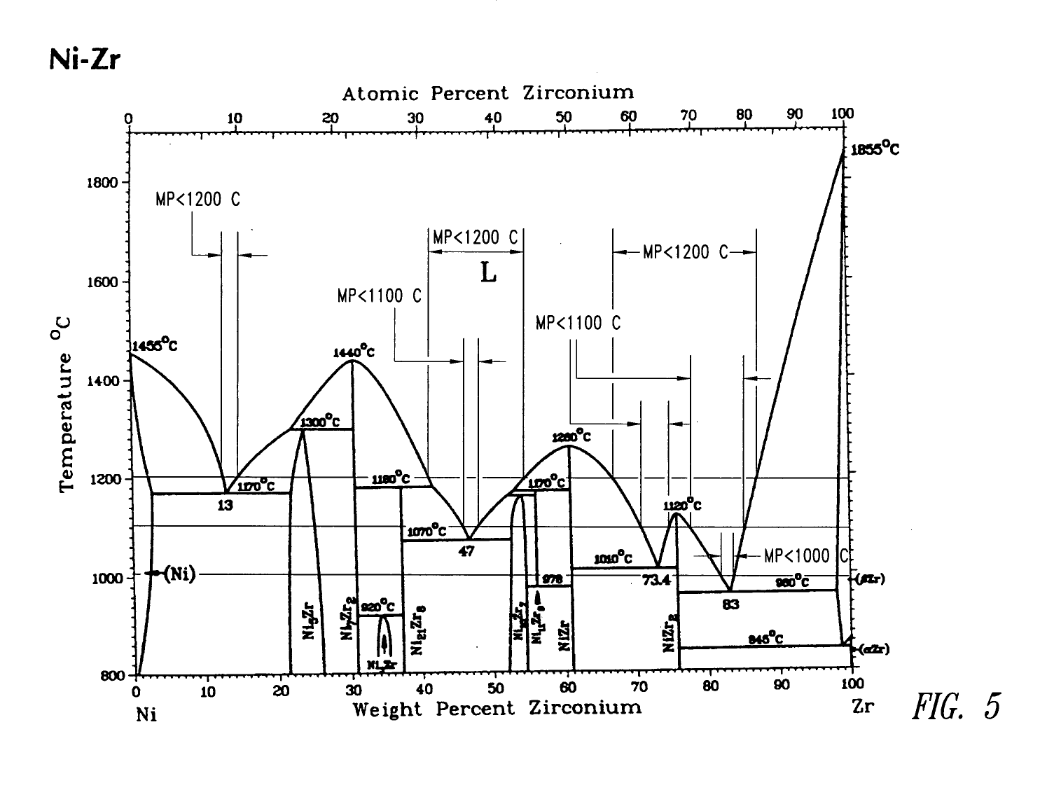

The binary phase diagram of nickel zirconium, illustrating compositions of the present invention, is shown in FIG. 5. It has a liquid-region boundary which contains four minima of melting point, each separated by higher-melting-point regions. These minima are:

960° C., at 83 wt % Zirconium, 17 wt % Nickel;

1010° C., at 73.4 wt % Zirconium, 26.6 wt % Nickel;

1070° C., at 47 wt % Zirconium, 53 wt % Nickel; and

1170° C., at 13 wt % Zirconium, 87 wt % Nickel.

All four of these low-melting-point regions have melting temperatures lower than 1200° C.

In the vicinity of the first of these points, melting points of less than 1000° C. are attained for compositions between approximately 81 wt % Zr (19 wt % Ni) and 84 wt % Zr (16 wt % Ni). In this same region of the phase diagram, melting points of less than 1100° C. are attained for compositions between approximately 77 wt % Zr (23 wt % Ni) and 85 wt % Zr (15 wt % Ni). In this same region of the phase diagram, melting points of less than 1200° C. are attained for compositions between approximately 67 wt % Zr (33 wt % Ni) and 87 wt % Zr (13 wt % Ni).

In the vicinity of the second of these points, melting points of less than 1100° C. are attained for compositions between approximately 70 wt % Zr (30 wt % Ni) and 75 wt % Zr (25 wt % Ni). In this same region of the phase diagram, melting points of less than 1200° C. are attained for compositions between approximately 67 wt % Zr (33 wt % Ni) and 87 wt % Zr (13 wt % Ni), which is the same composition range highlighted previously.

In the vicinity of the third of these points, melting points of less than 1100° C. are attained for compositions between approximately 46 wt % Zr (54 wt % Ni) and 48 wt % Zr (52 wt % Ni). In this same region of the phase diagram, melting points of less than 1200° C. are attained for compositions between approximately 41 wt % Zr (59 wt % Ni) and 54 wt % Zr (46 wt % Ni).

In the vicinity of the fourth of these points, melting points of less than 1200° C. are attained for compositions between approximately 12.5 wt % Zr (87.5 wt % Ni) and 14 wt % Zr (86 wt % Ni).

If it is adequate to have a melting point which is merely less than 1300° C., then the composition can be in the range from approximately 10 wt % Zr (90 wt % Ni) to approximately 22 wt % Zr (78 wt % Ni) or from approximately 38 wt % Zr (62 wt % Ni) to 89 wt % Zr (11 wt % Ni).

The composition of the present invention may contain nickel and zirconium in any of the ranges of relative proportions just listed. In addition to the use of alloys containing only nickel and zirconium in the stated proportions, it is also possible to use alloys comprising nickel and zirconium in the relative proportions as just described, together with a quantity of another constituent(s).

Compositions Containing Nickel and Niobium

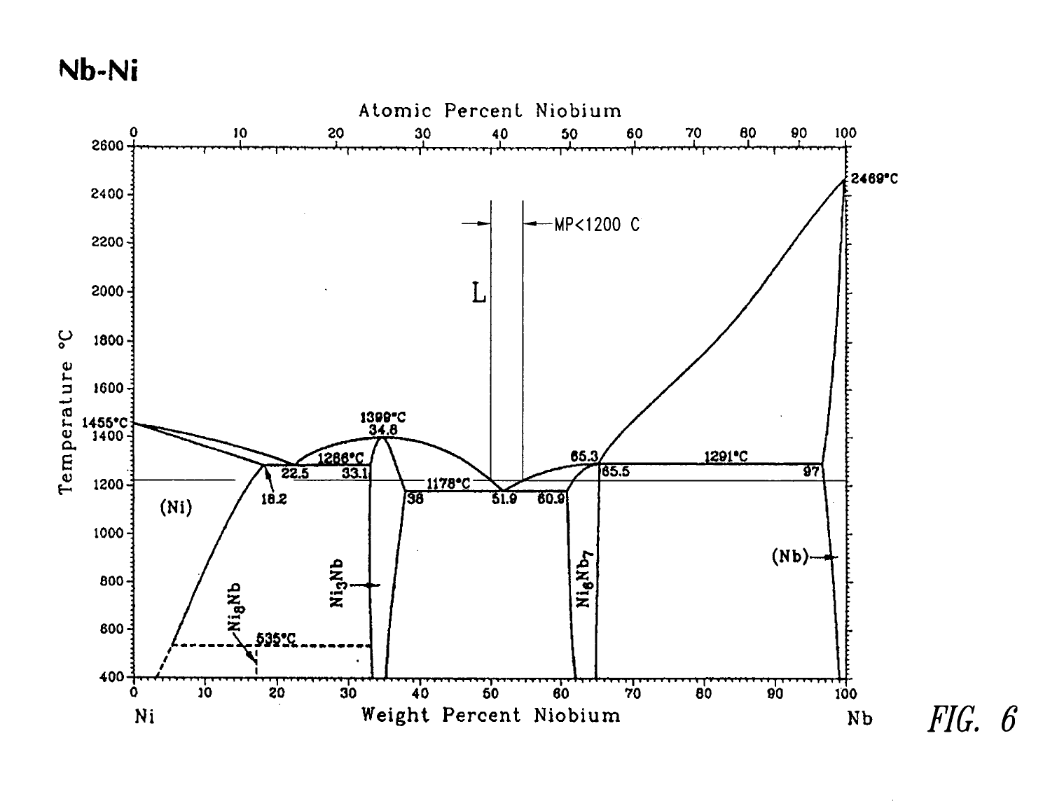

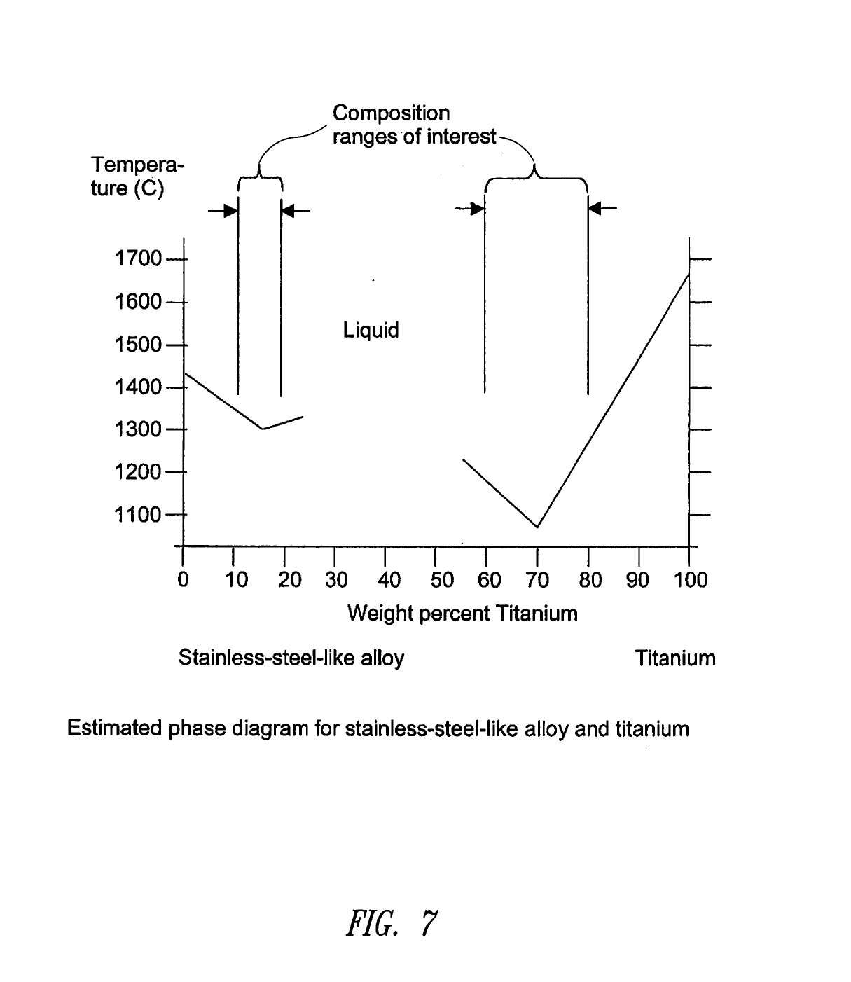

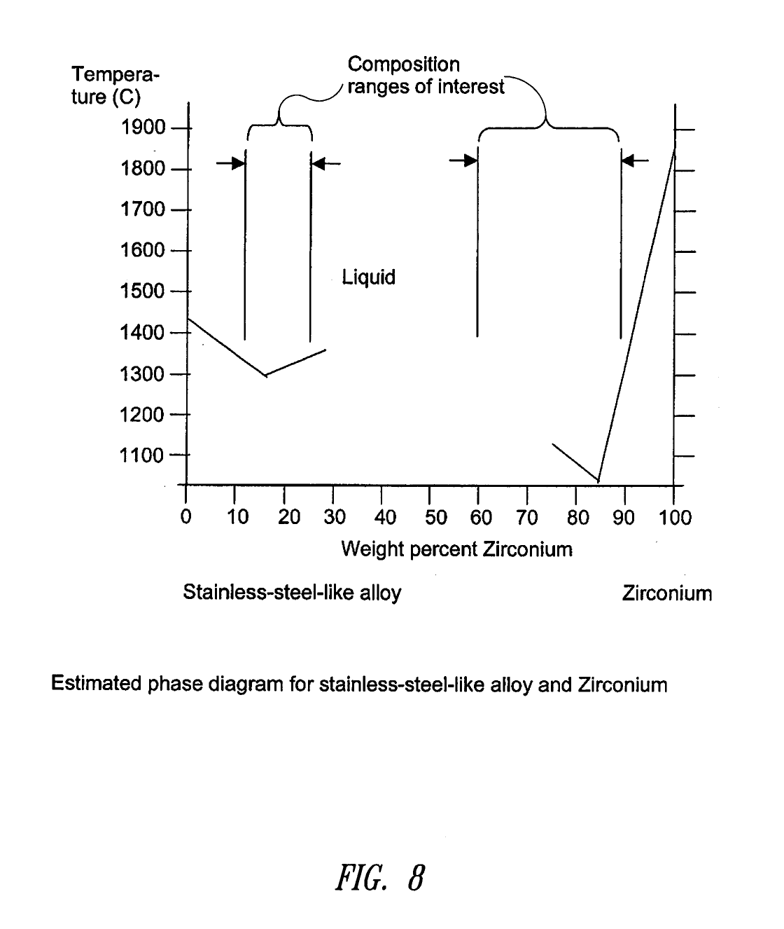

The binary phase diagram of nickel and niobium, illustrating compositions of the present invention, is shown in FIG. 6. It has a liquid-region boundary which contains three inflection points or minima of melting point. Of these three points, one of them is a melting point lower than 1200° C. It is 1178° C., at 51.9 wt % Niobium, 48.1 wt % Nickel.

In the vicinity of this point, melting points of less than 1200° C. are attained for compositions between approximately 51 wt % Nb (49% Ni) and 53 wt % Nb (47 wt % Ni).

If it is adequate to have a melting point which is merely less than 1300° C. then the composition can be in the range from approximately 46 wt % Nb (54 wt % Ni) to 65 wt % Nb (35 wt % Ni).

The composition of the present invention may contain nickel and niobium in any of the ranges of relative proportions just listed. In addition to the use of alloys containing only nickel and niobium in the stated proportions, it is also possible to use alloys comprising nickel and niobium in the relative proportions as just described, together with a quantity of another constituent(s).

Compositions Containing Stainless-steel-like Alloys, Together with Titanium, Zirconium or Niobium

Stainless steel is not an element or even a single precisely defined alloy. Rather, it is a family of alloys having the concentrations of major constituents within certain ranges. Stainless steel may be defined as any steel with a chromium content of greater than 10% by weight. Another constituent present in significant amounts in most stainless steels is nickel. Other minor alloying elements which appear in some stainless steels include vanadium, molybdenum, manganese, cobalt, silicon and phosphorus, carbon, nitrogen, titanium and niobium. The balance (and largest component) of stainless steel is iron.

As a summary of the entire family of stainless steels, it can be considered that the compositions of the present invention which refer to a stainless-steel-like alloy as a quasi-constituent include within the stainless-steel-like alloy any relative iron-chromium-nickel contents defined by the range of any chromium content from 10% to 30%, any nickel content from 0% to 20%, and an iron content greater than 50% which makes up the rest of the composition not occupied by chromium and nickel. These composition ranges encompass the nickel and chromium contents of substantially all commercial alloys of stainless steel.

Compositions of the present invention include alloys in which iron, chromium and nickel are present in the relative proportions just described, forming the stainless-steel-like alloy that is a quasi-constituent, together with at least one other element. In other words, this could be considered as alloying stainless steel (even though stainless steel is not an element) with at least one other element.

In order to estimate properties of such compositions, it is possible to combine binary phase diagrams individually involving iron, nickel and chromium, to the extent that those binary phase diagrams have similarities to each other, to provide an estimate of a pseudo binary phase diagram for stainless steel and the other constituent.

First, consider compositions containing mostly stainless steel with relatively small concentrations of titanium, as shown in FIG. 7. In this sense, titanium can be viewed as a melting point depressant. In the binary phase diagrams for iron-titanium, nickel-titanium and chromium-titanium, at the low-titanium end of the phase diagram, the slopes of melting point depression as a function of the concentration of Ti are, as follows: for Fe—Ti, 18° C./percent Ti; for Cr—Ti, 9° C./percent Ti; for Ni—Ti, 11° C./percent Ti. The average of these, weighted by the compositions of each component in stainless steel, is around 15° C./percent Ti.

The temperatures of the local minima of melting point at the low-titanium end of the phase diagram are as follows: for Fe—Ti, 1289° C. at 14 wt % Ti; for Cr—Ti, 1410° C. around the middle of the composition range; for Ni—Ti, 1304° C. at 14 wt % Ti. It can be expected that an effective eutectic for titanium with stainless-steel-like alloy would occur at a composition of around 85% stainless-steel-like alloy, 15% titanium. Compositions of interest for the present invention can include from 12% titanium 88% stainless-steel-like alloy to 20% titanium 80% stainless-steel-like alloy.

Next, consider compositions containing mostly stainless-steel-like alloy with relatively small concentrations of zirconium, as shown in FIG. 8. In this sense, zirconium can be viewed as a melting point depressant. Fe—Zr has a eutectic which is at 85% Fe, 15% Zr, having a melting temperature of 1337° C. Cr—Zr has a eutectic which is at 72% Cr, 28% Zr, having a melting temperature of 1592° C. Ni—Zr has one of its eutectics at 87% Ni, 13% Zr, having a melting temperature of 1170° C.

So all three of these stainless-steel-like alloy constituents have a eutectic for a zirconium content somewhere in the teens or 20's percent. Accordingly, it can be estimated that there is a eutectic for zirconium plus stainless-steel-like-alloy at a composition somewhere around 18% zirconium 82% stainless-steel-like alloy. Corresponding to this effective eutectic, a composition range of interest for the present invention would be from about 15% zirconium 85% stainless-steel-like alloy to about 25% zirconium 75% stainless-steel-like alloy.

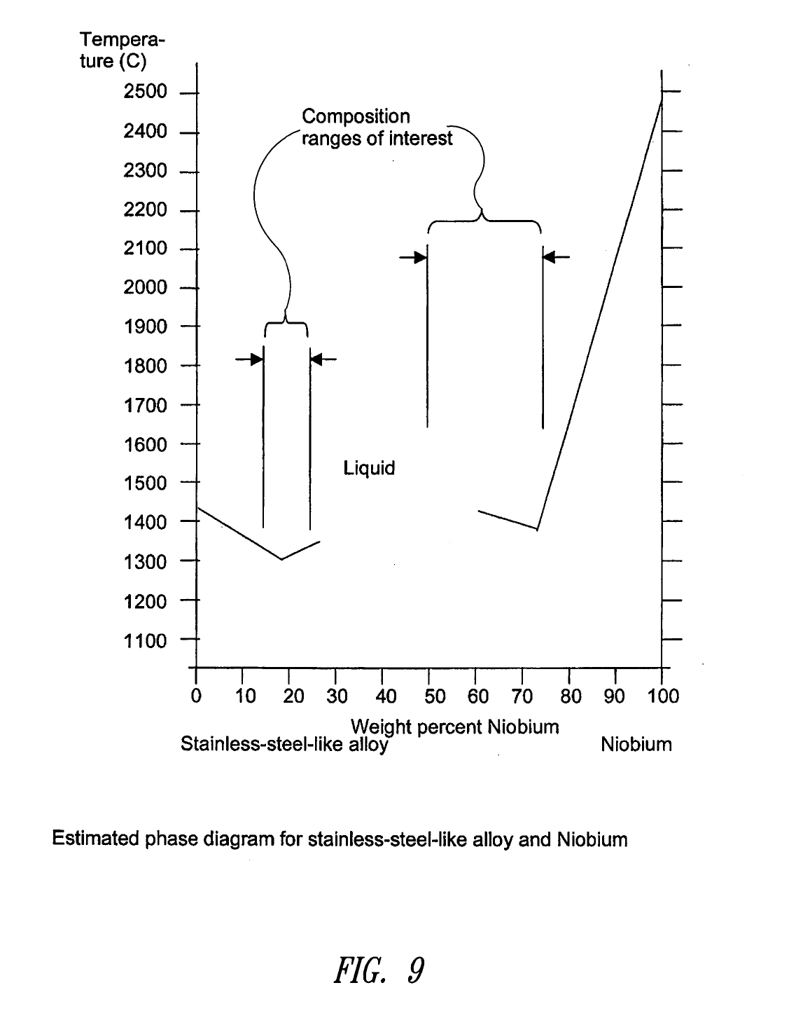

Next consider compositions containing mostly stainless steel with relatively small concentrations of niobium, as shown in FIG. 9. Cr, Fe and Ni each has a eutectic with niobium at around 19% niobium. The niobium can be viewed as a melting point depressant. The same patterns as above are observed but the melting points are probably not reduced as far as what is obtained using titanium or zirconium. Compositions of interest for the present invention can be from 15% niobium 85% stainless-steel-like alloy to 25% niobium 75% stainless-steel-like alloy.

Next consider the same combinations of stainless steel with the three mentioned elements at the other end of the composition range, in which the concentration of stainless steel is a minority. First consider titanium as a majority constituent, with stainless steel as a minority constituent, which is shown in FIG. 7. For mostly Ti containing a minority amount of stainless-steel-like alloy mixed in: Fe—Ti has a eutectic of 1085° C. at 70% Ti, 30% Fe. Cr—Ti has a minimum liquid temperature of 1410° C. near the mid-point of composition. Ni—Ti has a eutectic of 942 C at 28% Ni 72% Ti. So, it can be expected that at about 70% Ti about 30% stainless-steel-like alloy there would be a melting point somewhere less than 1100 C or certainly well below 1200 C, as shown in FIG. 7. So, compositions of interest for the present invention may include from 60% titanium 40% stainless-steel-like alloy to 80% titanium 20% stainless-steel-like alloy.

Next consider zirconium as a majority constituent, with stainless-steel-like alloy as a minority constituent, as shown in FIG. 8. Zr—Fe has a eutectic at 928 C for 16% Fe, 84% Zr. Zr—Cr has a eutectic at 1332 C for 14% Cr, 86% Zr. Zr—Ni has a eutectic at 960 C for 17% Ni, 83% Zr. All three of these behaviors are really quite similar to each other. Based on this, and trying to obtain some sort of representative average combining the behavior of Fe and Ni and Cr, we can expect that a composition of about 15% stainless-steel-like alloy, 85% Zr would have a eutectic somewhere around 1000 C or certainly well below 1200 C. Compositions of interest for the present invention may include from 60% zirconium 40% stainless-steel-like alloy to 90% zirconium 10% stainless-steel-like alloy.

Finally, consider niobium as a majority constituent, with stainless-steel-like alloy as a minority constituent, as shown in FIG. 9. There are eutectics of Nb—Fe at 75% Nb, Cr—Nb at 64% Nb, and Nb—Ni at 52% to 65% Nb. An effective eutectic for Nb with a stainless-steel-like alloy may be expected at a composition of around 65% Nb, and compositions of interest for the present invention may include from 50% niobium 50% stainless-steel-like alloy to 75% niobium 25% stainless-steel-like alloy.

The above examples have considered individually mixing Zr or Ti or Nb with stainless steel. It would also be possible to consider adding two or even all three of Zr, Ti and Nb simultaneously to stainless steel. At the end of the composition range which is mostly stainless-steel-like alloy, compositions of interest could be anywhere from 88% stainless-steel-like alloy to 75% stainless-steel-like alloy, with the balance being any combination of titanium and zirconium and niobium. At the end of the composition range which is a minority of stainless-steel-like alloy, compositions of interest could be anywhere from 40% stainless-steel-like alloy to 10% stainless-steel-like alloy, with the balance being any combination of titanium and zirconium and niobium.

other Alloys Based on Known Ternary Phase Diagrams

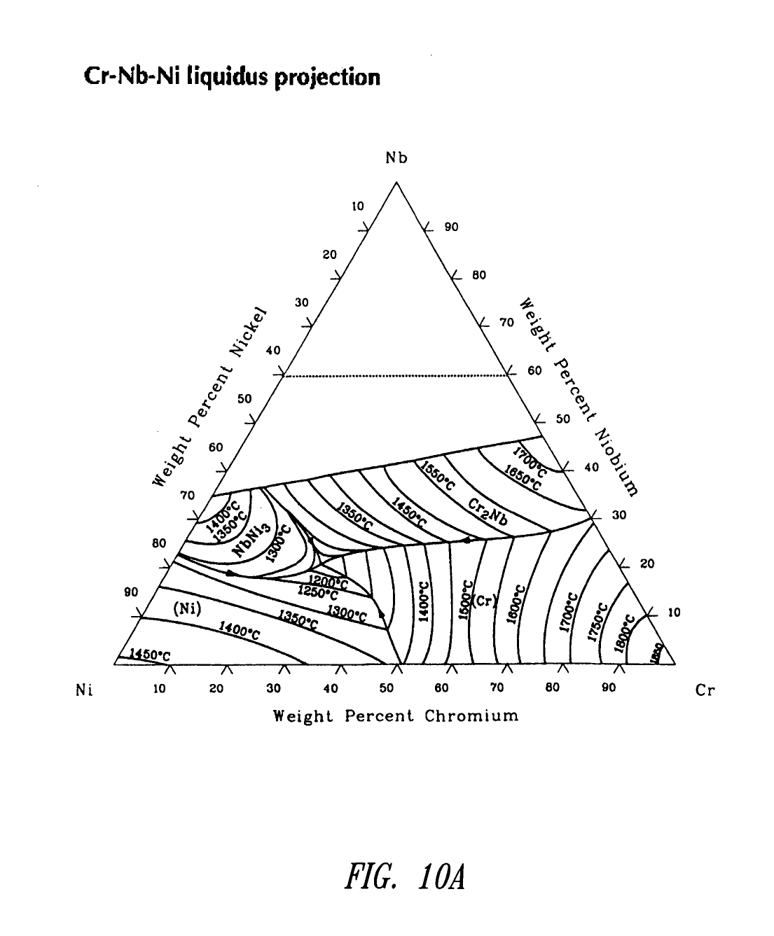

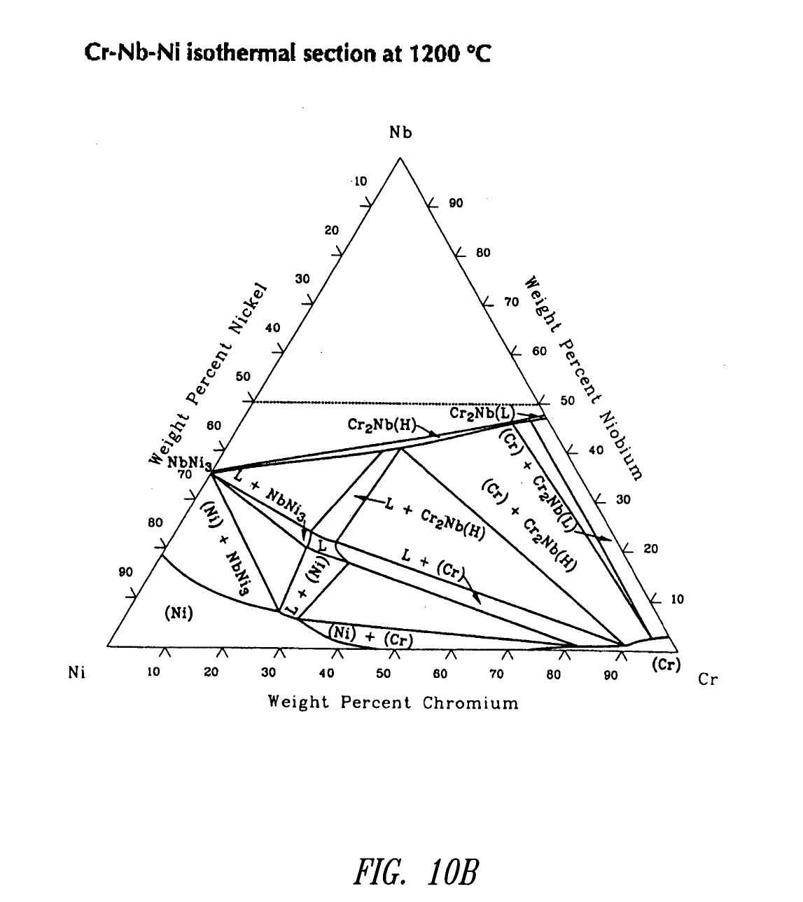

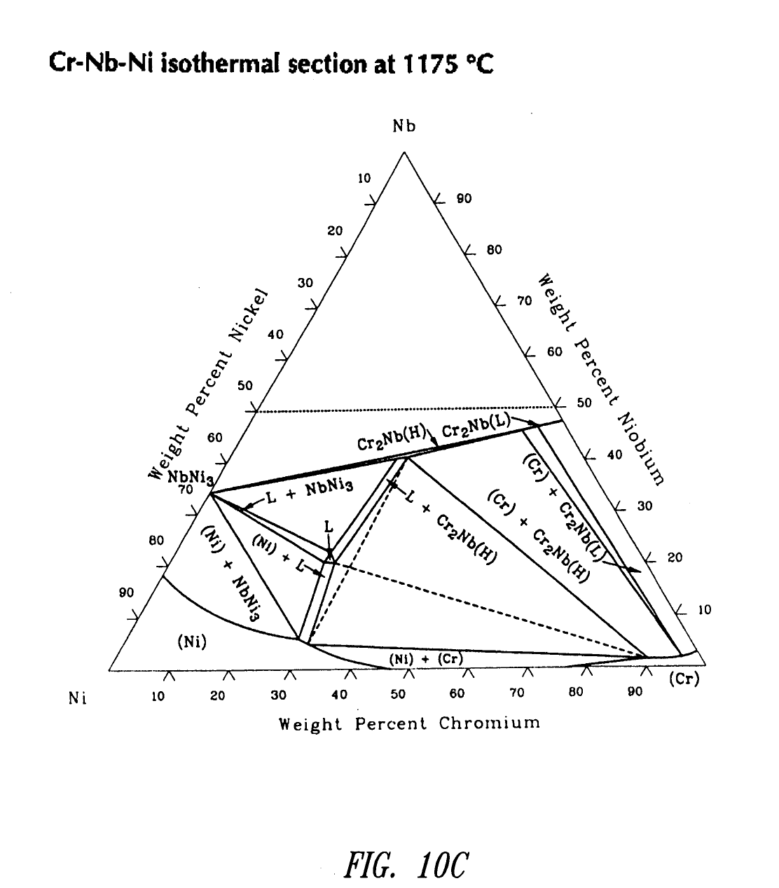

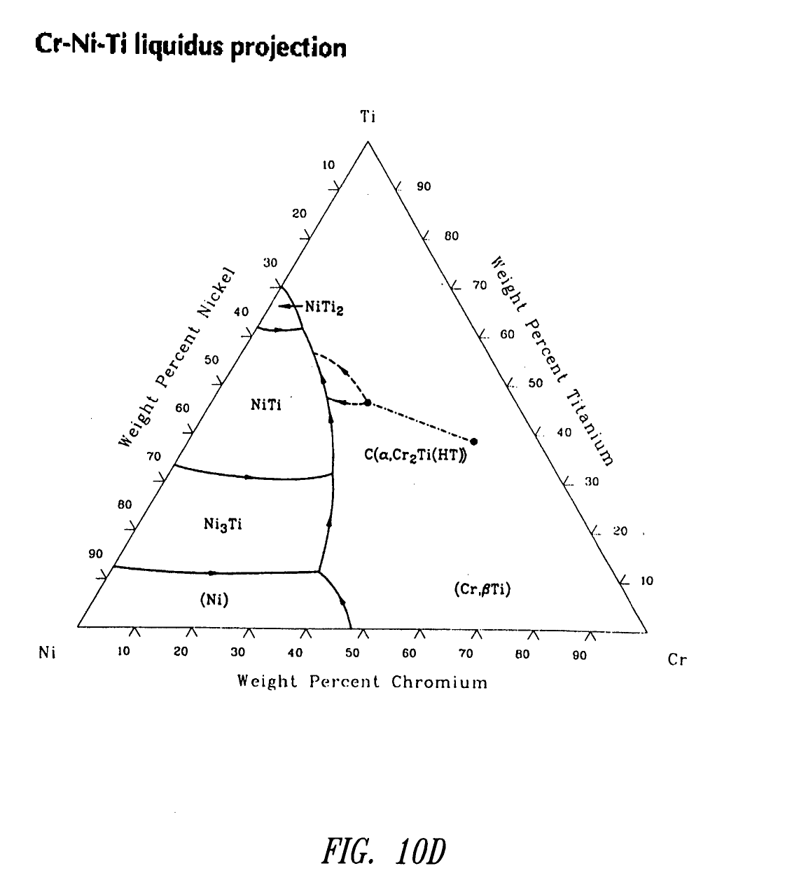

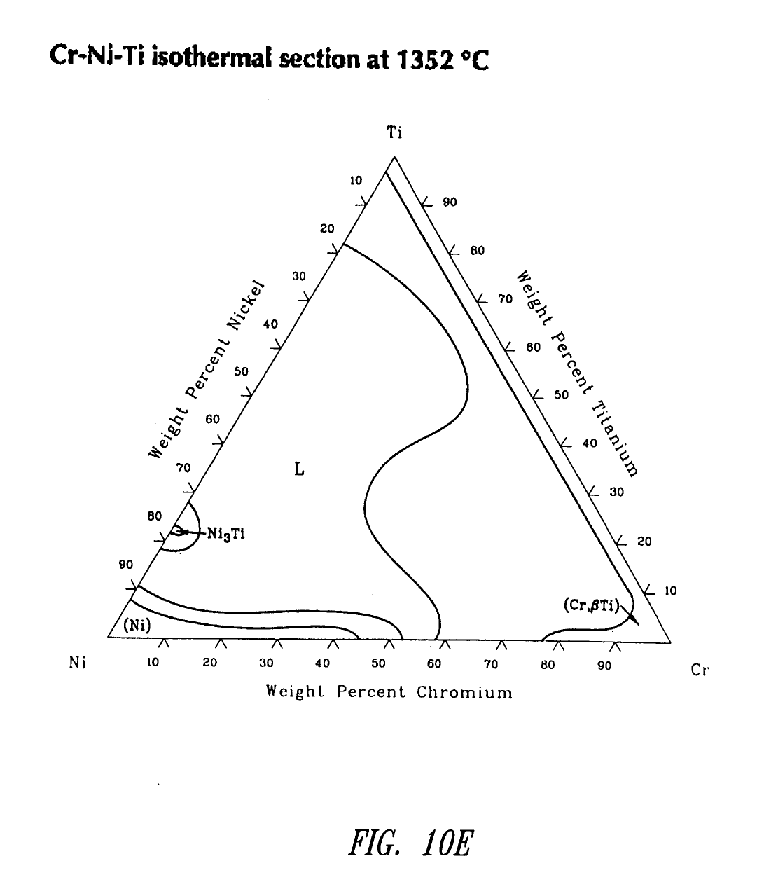

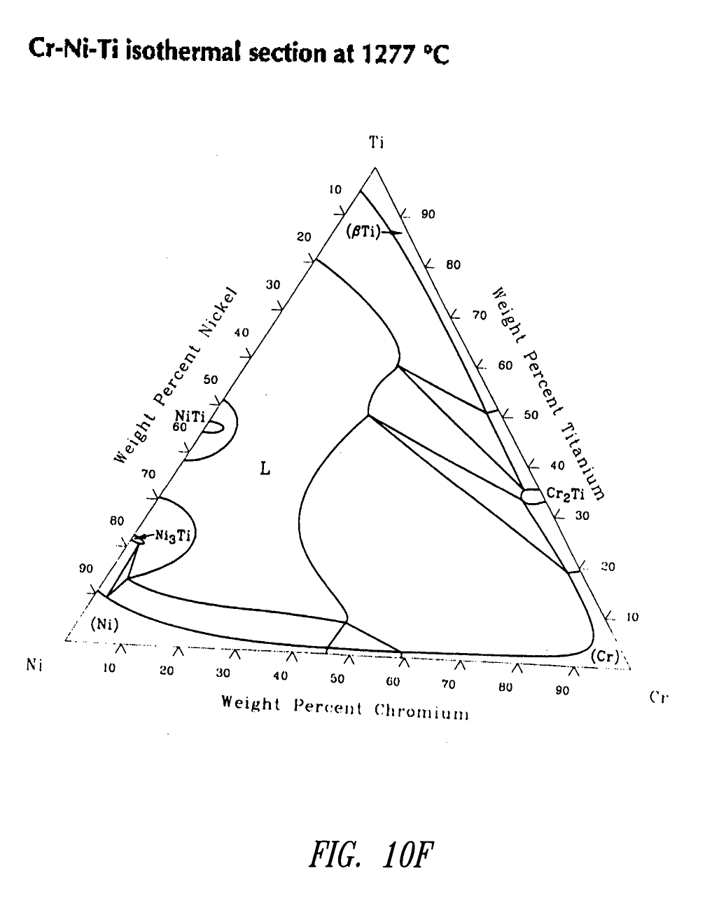

In ternary alloy diagrams (which are available in the literature for only a very few of the many possible combinations), there are several interesting diagrams and regions. These are shown in FIGS. 10A-10G.

As shown in FIG. 10G, at 1027° C. the chrome-nickel-titanium diagram has a small region of liquid at high titanium content resembling the Ni—Ti binary phase diagram (35% Ni, 65% Ti, small concentration of Cr such as less than 10%). As shown in FIG. 10F, at a temperature of 1277° C., the chrome-nickel-titanium diagram has liquid down to a quite small titanium content around 10% Ti (balance being from half-nickel half-chromium to mostly-nickel). This probably implies that some liquid region persists down to lower temperatures such as 1200° C. As shown in FIG. 10C, at a temperature of 1175° C., the chrome-nickel-niobium diagram has a small region of liquid at about 20% Nb, 30% Cr, 50% Ni. The composition of the present invention could be chosen to be, at a desired temperature, within the labeled liquid regions of any of these ternary phase diagrams in FIGS. 10A-10G.

Transient Liquid Phase Sintering

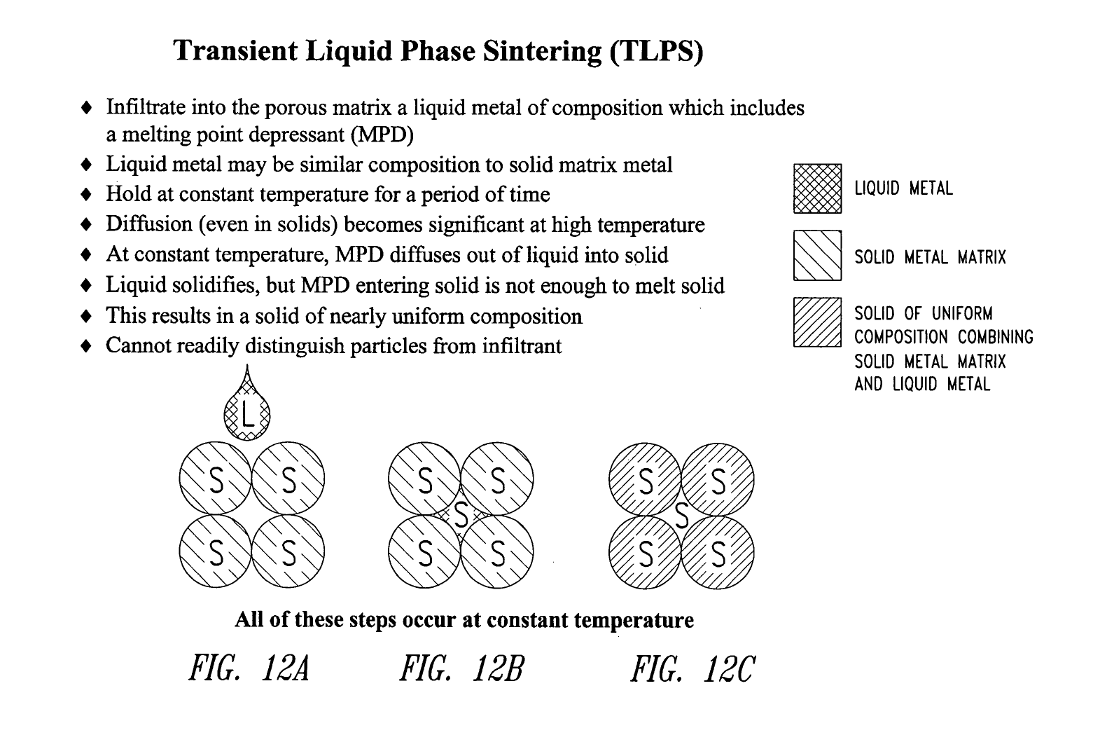

As illustrated in FIGS. 12A-12C, Transient Liquid Phase Sintering (TLPS) is possible if both the matrix and the infiltrant are metals. In Transient Liquid Phase Sintering, the infiltrating liquid metal can be a metal such as nickel containing a melting point depressant (MPD). In an especially simple case, the powder or matrix can be similar to the composition of the infiltrant except that the powder or matrix would not contain the melting point depressant contained in the infiltrant. Then, when the infiltrant is at the infiltrating temperature and is liquid, the liquid enters the porous metal powder or matrix and the part can be maintained for a while at that same infiltrating temperature.

During this period of time the melting point depressant can diffuse out of the infiltrant liquid into the solid metal particles making up the part. Eventually, the amount of melting point depressant remaining in the infiltrated liquid becomes small enough that the infiltrant becomes solid. The amount of melting point depressant entering the solid particles may be insufficient to cause the solid particles to melt. So, the liquid infiltrant can solidify while not experiencing any change of temperature, simply by the action of diffusion of the melting point depressant substance out of the liquid into the powder particles. The article can be held at elevated temperature long enough and at appropriate temperature so that diffusion essentially equilibrates the composition of the original infiltrant material and the composition of the original solid particles, especially in terms of the concentration of the melting point depressant. The idea is that this will result in a part that is essentially all one material and no distinction will remain between what was originally solid particle material and what was originally infiltrant.

The Transient Liquid Solidification Process can be performed using the low melting point biocompatible alloys of the present invention as infiltrants. Doing this with the infiltrant compositions described herein should result in an article whose composition is biocompatible, and also whose composition is close to uniform throughout.

This can be a way of three dimensionally printing and manufacturing solid parts of uniform or almost uniform composition out of metal compositions which are biomedically useful, something which has not been achieved anywhere in the literature yet.

Processing Options

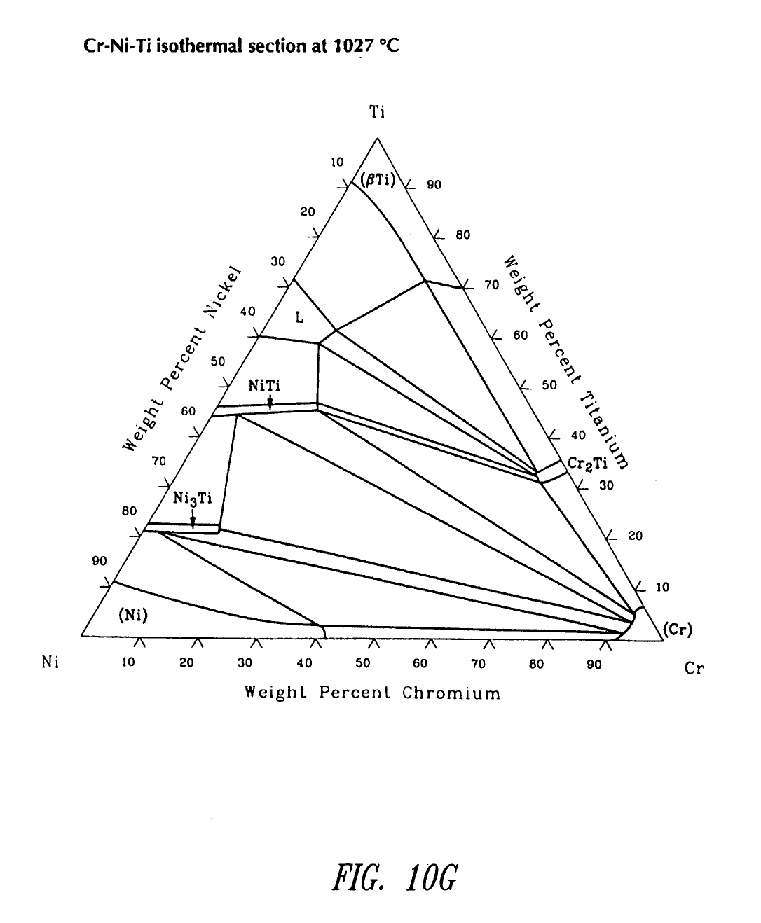

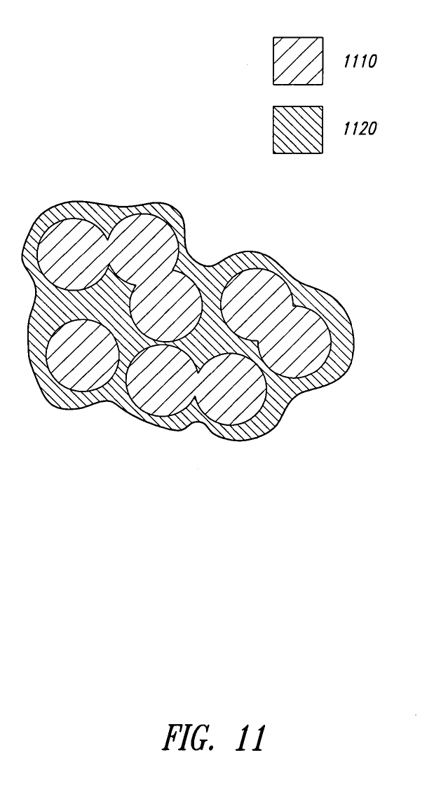

One way of using low melting point biocompatible metal alloys is to print and then partially sinter an article made entirely of a powder having a relatively high-melting-point, and then expose the article to a melt (molten bath) of the infiltrant alloy and allow the melt to wick into the article by capillary action. For this purpose, the article and the molten infiltrant may be prepared to both be at temperatures (either equal or different) which are above the melting point of the infiltrant composition. This final configuration is illustrated in FIG. 11. Ceramic particles 1110 may be partially sintered to each other. An infiltrant composition 1120 according to aspects of the present invention occupies the space between the ceramic particles 1110.

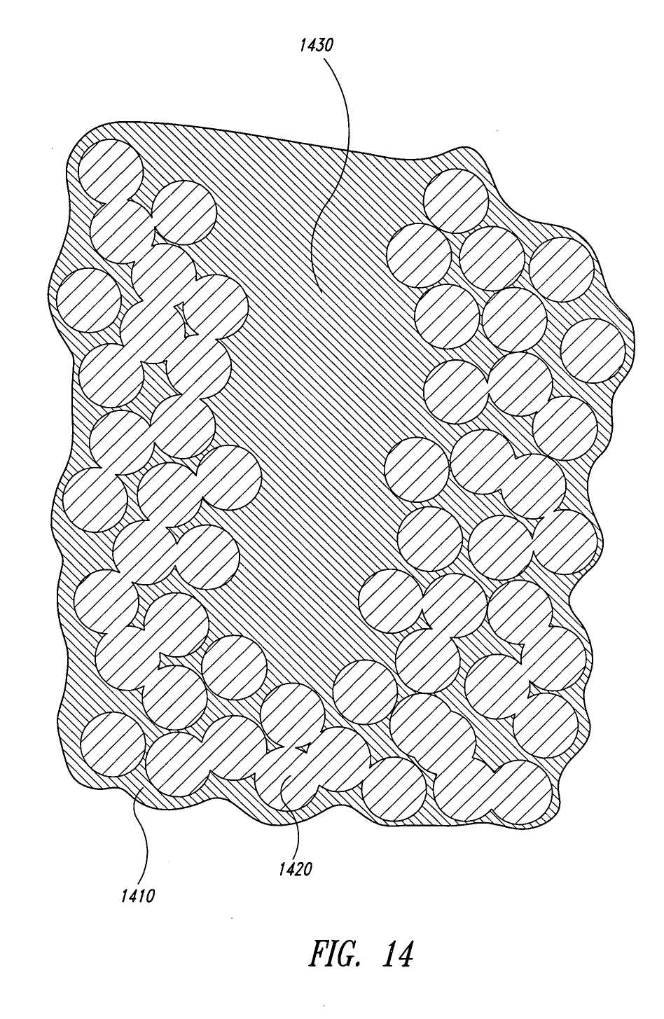

As illustrated in FIG. 14, and discussed further herein, a low melting point metal infiltrant 1410 could also be cast into a matrix 1420 or other porous article, which may involve designing in channels 1430, cavities, runners, sprues and the like.

These are still not the only possible way of introducing an infiltrant or liquid into a porous article. It is also possible that the low melting point (infiltrant) alloy be prepared as a powder and incorporated into the printed article as part of the 3DP process. For example, during the manufacturing steps that precede sintering, the powder particles of the main powder material are usually joined to each other by a binder substance, and eventually that binder substance decomposes and exits and then the powder particles sinter to each other. It is entirely possible that powder particles of the infiltrant material could be joined to each other and to the powder particles of the main material during the 3DP process, such as by the same binder substance. They would be with the rest of the part when the part enters an oven for binder burnout and for sintering.

Temperatures and melting points may be chosen so that it is possible to sinter the particles of the main powder material at some temperature, which is less than the melting point of the infiltrant. Of course, since the infiltrant would be even closer to its melting temperature than the main powder particles would be to their melting temperature, sintering could be expected to occur in the infiltrant particles to an even greater degree, but the infiltrant particles would not be able to actually melt because the temperature would be below their melting temperature. Then, after sintering of the main powder particles has occurred, the oven temperature could be increased enough to melt the infiltrant and let it wick or flow into the porous matrix of the main part which has by now formed a porous sintered matrix.

The infiltrant, in the form of powder, would be somewhere in the 3DP part or on the surface of the 3DP part, ready to melt when heated to an appropriate temperature. The meltable infiltrant powder could be contained in certain layers such as a layer which bounds an external plane of the printed part, especially if the part had at least one flat surface which could be arranged to coincide with a layer in three dimensional printing. In order to accomplish this, it would be possible to switch powders in between layers of 3DP. In a bit more detail, different powders could be placed, prior to printing of binder liquid in 3DP, in stripes within powder layers or in even more specific geometries and locations, before dispensing of binder liquid onto the powder in the 3DP process.

Processing temperatures could be chosen with a first hold-temperature so that the base powder sinters (and the meltable metal powder sinters more), followed by a higher temperature that causes the meltable metal alloy powder to melt and flow into the pores of the other sintered powder. The main powder particles could be ceramic or they could be a metal having a melting point higher than the melting point of the infiltrant.

Article of Manufacture: Implant Containing Metal Composition Plus Ceramic

An aspect of the invention is an article manufactured which includes the metal composition of the invention. The article can include a first network which defines a second network which interlocks with the first network. The second network may be at least partly filled with the composition of the present invention.

The first network may be or may include a ceramic, and in particular may be or may include a resorbable ceramic, which may be or may comprise a significant fraction of tricalcium phosphate such as beta tricalcium phosphate. This would result in a bone substitute implant which has at least some modest amount of mechanical strength at the time it is implanted, and which also has to ability to become integrated with natural bone because the resorbable ceramic can be replaced by natural bone as it resorbs, and the metal network can remain in place but become enmeshed in and surrounded by natural bone which grows in to replace the resorbed ceramic. Alternatively, the first network may be or may include a nonresorbable ceramic of the calcium phosphate family such as hydroxyapatite. The first network may include still other ceramics such as zirconia or alumina. The first network may be or may include bioglass.

Alternatively, the first network may be or may include a metal. The metal of the first network may be different from the metal composition of the infiltrant, or may have overlap in composition with the metal composition of the infiltrant.

The invention also includes articles having channels, cavities, sprues, runners etc. (i.e. regions which are more macroscopic than pores) filled with the metal composition of the present invention.

Articles of the present invention which comprise a metal first network infiltrated by the metal composition of the present invention could be articles such as surgical tools (e.g. endoscope parts), or they could be implantable articles.

Articles of the present invention could be implantable parts. They could be reinforcements or fillers for bone voids in non-load-bearing parts of the skeleton or load-bearing parts of the skeleton such as limbs and extremities. They could be spinal cages, and could be a spinal cage whose interior is already filled without requiring a physically separate spinal cage insert. Such an article could contain an interior region which comprises metal-infiltrated ceramic, with the interior region being surrounded in at least some places by an exterior region which is essentially solid metal. The ceramic network in the interior region could be or could include resorbable ceramic such as tricalcium phosphate. The metal network in the interior region and the essentially solid metal in the exterior region could be integrally joined to each other as a result of having solidified from liquid at essentially the same time.

Selective Infiltration of Metal into Porous Matrix

In some preferred embodiments of this invention, it is desirable to fill only part of the pore space of the starting matrix material with metal infiltrant, leaving an outer porous layer of the original matrix. Such an approach is advantageous for matrix materials that are more biocompatible or bioactive than the infiltrant.

One illustration of an article with only partial infiltration is shown in FIG. 16. Particles of a first network material 1620 such as a ceramic, metal or bioglass, are partially infiltrated with a metal infiltrant 1610. Space 1630 that did not receive an infiltrant may remain empty, or may be later filled with a bioactive substance.

As an example, matrices of bioactive ceramics such as hydroxyapatite, tricalcium phosphate, or bioactive glass may be infiltrated selectively with titanium/nickel alloys. For such devices, an outer porous layer of hydroxyapatite, tricalcium phosphate, or bioactive glass would significantly improve the bone-bonding capability of the device, while the infiltrated metal provides a vast improvement in the load-bearing capability.

A preferred approach to selective infiltration involves characterization of the available pore space in the starting matrix and addition of a metered or controlled amount of infiltrant. This is described in detail in co-pending U.S. Application Nos. 60/467,474; 60/488,362; and 60/486,404 herein incorporated in their entirety by reference.

Starting matrices may be characterized using such techniques as mercury infiltration, the results of which provide a total available pore volume. Based on this pore volume and the true density of the infiltrant in the solid state, it is possible to weigh out the desired amount of metal infiltrant required to reach a target fill percentage in the pore space of each device. This known (i.e., weighed) mass of infiltrant material may then be loaded into a furnace in such a way that it is in contact with the characterized porous matrix.

Upon firing and melting of the infiltrant, there will be a natural tendency of the material to “wick” into the porous matrix due to the surface tension of the infiltrant. This mechanism of capillary action will also tend to distribute the infiltrant preferentially in the smallest available pores provided that there is no gas entrapment. Upon cooling, the infiltrant will solidify in the pores and yield a device that is a composite of the original matrix and infiltrant in some regions, and simply porous matrix in others. In general, the capillary-based mechanism will yield porous regions toward the exposed surfaces of the device.

Using a process such as three-dimensional printing, a starting porous matrix can be made with porosity at multiple scales. By controlling binder droplet placement, “negatively printed” features (i.e., channels) can be created in the order of 100 microns in size. Conversely, in the printed regions, the inherent porosity from the starting powder falls in size ranges from 1-50 microns, depending on print parameters. Thus, it is possible to create porous matrices with multiple populations of porosity at different orders of magnitude in size, such as 1, 10, or 100 microns.

Selective infiltration of such a porous matrix may be used to fill the smallest pores preferentially with infiltrant, while leaving the largest pores open for tissue ingrowth, apposition, or attachment. In the case of a hydroxyapatite, tricalcium phosphate, or bioglass starting matrix that has been partially infiltrated with metal, it may be possible to retain large scale pores or channels (on the order of 100 microns) that traverse the entire implant end to end, while filling the smaller pores. If the quantity of infiltrant is small enough, the surfaces of these large pores or channels will consist solely of the original matrix material, improving the tendency to integrate with bone or other tissue.

Metal infiltration is distinguished from polymer infiltration in that the temperatures are significantly higher, and it is often necessary to carry out the heating step under a controlled atmosphere or vacuum to prevent the undesired oxidation of the metal. Vacuum is also useful to prevent the entrapment of gases within the pore space of the matrix during infiltration.

Introduction of Porosity to Matrix-infiltrant Composites after Infiltration

In some embodiments of this invention, it may be desirable to create porosity after the infiltrant has been added to the starting porous matrix. One way to achieve this is to include a chemical species in the starting matrix that has a substantially higher solubility in a suitable solvent than the other materials present in the final device.

In one embodiment, a starting matrix of beta-tricalcium phosphate and dicalcium phosphate may be infiltrated with titanium/nickel alloy. At neutral to alkaline pH, the aqueous solubility of dicalcium phosphate is an order of magnitude greater than that of beta-tricalcium phosphate, allowing for the preferential dissolution of dicalcium phosphate from the matrix.

In another embodiment, it may be desirable to introduce a more soluble ion into the initial matrix material to yield a more soluble species for dissolution after infiltration with metals. These ions may include highly-soluble, monovalent ions such as Na+ and K+.

In still another embodiment, it may be desirable to choose starting powders (for the fabrication of the initial porous matrix) that undergo chemical reactions in order to yield the more soluble and less soluble species described above.

In yet another embodiment, it may be desirable to introduce one or more chemical species via three-dimensional printing that then facilitate chemical reaction in order to yield the more soluble and less soluble species described above.

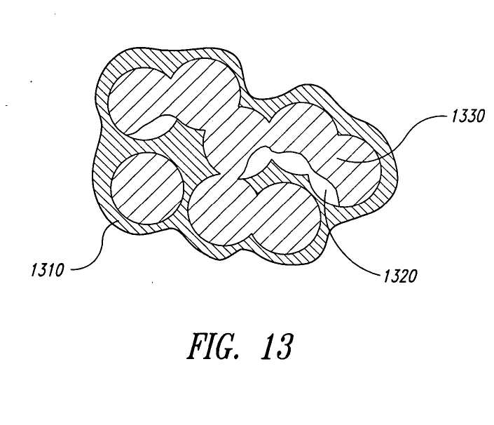

Some of the chemical species that may be incorporated into the starting matrix include sodium phosphate and potassium phosphate. These materials may be blended as powders with other materials of interest (tricalcium phosphate, hydroxyapatite, bioactive glass, or other bioceramics) prior to three-dimensional printing and sintering. The resulting porous matrix will then contain chemical species with substantially different solubility. After such a matrix has been infiltrated, in part or in full, the more soluble species may be leached to create additional porosity. This porosity may occur along the interface of the initial porous matrix and the infiltrant phases. This is illustrated in FIG. 13. An infiltrant composition of the present invention is shown between the particles 1330 forming the matrix; voids 1320 are illustrated. The voids 1320 result from removal of some of the particles 1330 after the infiltration of the composition 1310.

The use of chemical reaction to form an initial porous matrix is best illustrated by example. In one case, it may be desirable to start with a powder blend of dicalcium phosphate and sodium carbonate prior to three-dimensional printing and sintering. During the heating process, the sodium carbonate will decompose into sodium oxide (with consequent release of carbon dioxide), and may subsequently scavenge some of the phosphate ions. The dicalcium phosphate may then form tricalcium phosphate due to the loss of phosphate ions to sodium. Thus, a resulting composition of tricalcium phosphate (a less soluble species) and sodium phosphate (a more soluble species) may be obtained. After infiltration, the sodium phosphate may be dissolved preferentially, leaving behind the composite of tricalcium phosphate with infiltrant and some porosity.

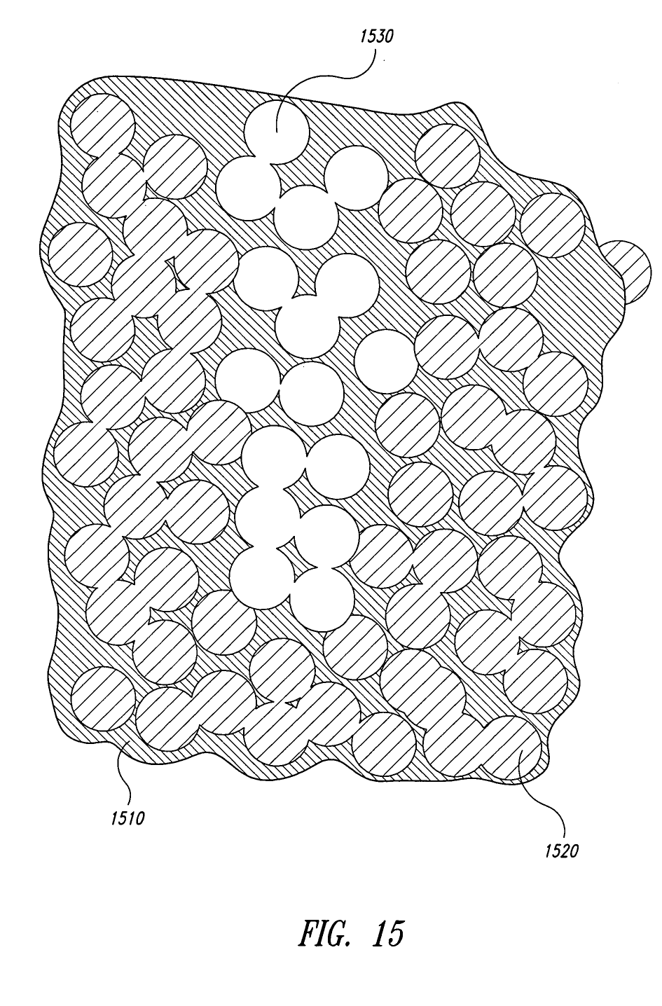

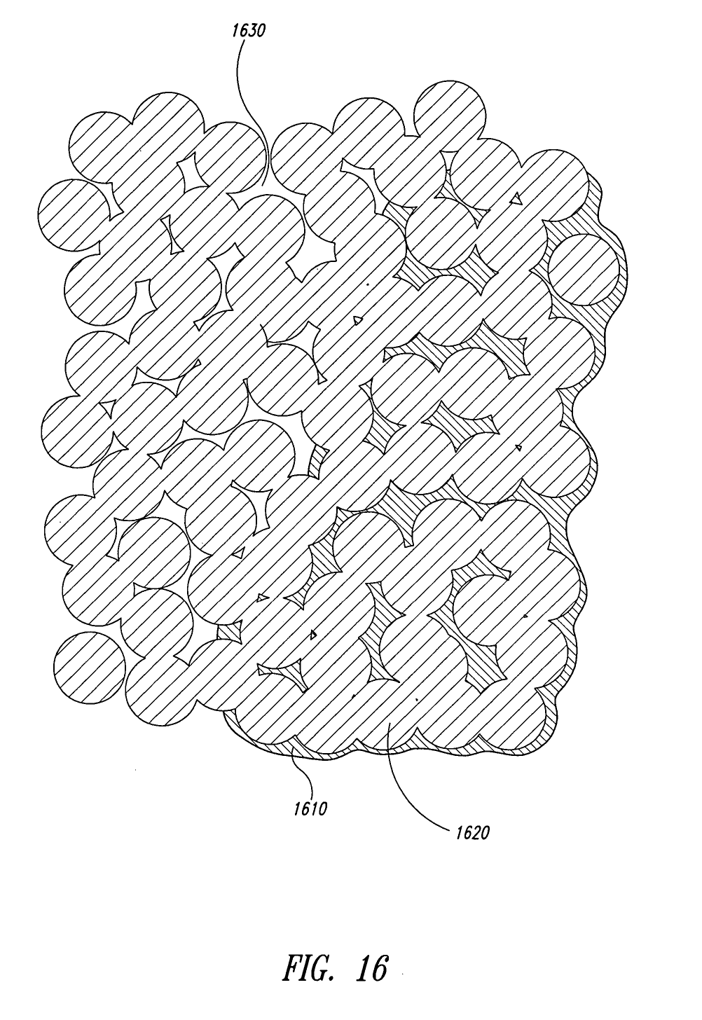

As illustrated in FIG. 15, particles of a first network 1520 and particles 1530 are infiltrated by a metal composition 1510. The particles 1530 are then preferentially dissolved such that a desired porosity remains.

In another case, chemical reaction may occur after selective deposition of one or more materials via three-dimensional printing. For example, materials such as sodium nitrate or potassium nitrate may be deposited onto a bioceramic (hydroxyapatite, tricalcium phosphate, bioglass, etc.) to yield more soluble species after firing. These nitrates provide a more soluble route of introduction for the Na+ and K+ ions than the carbonates mentioned in bulk powder blending above, and thereby enable three-dimensional printing as a method of combining reagents. Upon firing, the nitrates give off oxides of nitrogen as gas, leaving the Na+ and K+ behind to react and form highly soluble species.

The reverse approach (i.e., forming a less soluble species) is also valid. A material such as calcium nitrate may be introduced via three-dimensional printing in order to yield less soluble species after reaction than the initial powder material. For example, calcium nitrate may be printed onto dicalcium phosphate to chemically convert some of the material to beta-tricalcium phosphate or hydroxyapatite (both less soluble than dicalcium phosphate) after firing. Similarly, calcium nitrate may be added to sodium phosphate (or potassium phosphate) to generate a calcium phosphate of interest (dicalcium phosphate, tricalcium phosphate, hydroxyapatite) in addition to the original sodium phosphate (or potassium phosphate). In each of these cases, the highly soluble calcium nitrate is dissolved and printed in selected regions of the powder bed, forming the less soluble species after firing, and the remaining untreated powder comprises the more soluble species.

If the article at one of these late stages of manufacturing contains empty space such as pores, such as resulting from less-than-full infiltration by metal or resulting from dissolution of some of the non-metal material after infiltration by metal, it is possible for that empty space to contain a bioactive substance such as bone morphogenic protein, platelet rich plasma, etc. The presence of empty pores provides the ability to wick bodily substances such as blood, platelet rich plasma, etc., which can be advantageous.

In another aspect of the invention, the ability to control the composition of ceramic in different places within the article, such as by localized chemical conversion of one ceramic composition to another, makes it possible to make an article which has hydroxyapatite in some desired places, tricalcium phosphate in other desired places, and metal infiltrant either in all pores or in pores in selected places in the article.

Method Aspects and other Aspects of the Invention

The invention also includes methods of manufacturing, i.e., methods of infiltrating a metal infiltrant which use the biocompatible low melting point metal alloys described herein. The methods of the invention also include possibly casting the described compositions, so as to form an article which contains more macroscopic channels sprues runners etc. for the infiltrant to flow in, the channels sprues, runners, and the like, being built into the article, as opposed to infiltrating or in addition to infiltrating. This is illustrated in FIG. 14.

Forming an article which has some solid metal regions on its exterior can involve making a porous ceramic which eventually comprises the first network, such as by three-dimensional printing followed by sintering, and also making a mold to at least approximately define the exterior shape of the article. The mold could be made integrally with the first network or could be made as a separate piece. The mold could be made by the same process which was used to make the porous ceramic first network or could be made by a different process. Similarly, the material of the mold could be the same as or different from the material of the first network. After completion of casting and infiltrating, the mold could be removed such as by being broken. After the removal of the article from the mold or the mold from the article, there could be still other manufacturing operations performed on the article such as to further refine its external shape, such as to give it threads, serrations, etc.

The invention also includes articles manufactured by the described method. For example, the invention includes articles manufactured by manufacturing a porous matrix, such as by three-dimensional printing followed by sintering, followed by infiltrating with liquid metal and holding at a suitable temperature such that Transient Liquid Phase Sintering has blurred or eliminated the boundaries between particle and infiltrant.

Further Comments, Summary and Advantages

Except for sintering, with the consequent large dimensional change and associated dimensional inaccuracy of the part, the entire field of solid-metal parts made by 3DP for biomedical purposes has really still been impractical for lack of a biocompatible infusing metal. A biocompatible infusing metal could make metal-infused 3DP parts practical for both implants and for external devices such as endoscope components, and also in general for other applications where corrosion is a concern, either medical or non-medical.

The present invention allows manufacture of a ceramic-metal implant whose ceramic portion is resorbable. The metal portion provides strength allowing an implant which is capable of carrying at least some mechanical load during the healing process. The resorbability means that the ceramic portion will eventually be replaced by natural bone resulting in integration of the implant (or what remains of the implant, i.e., the metal portion) into natural bone.

A preferred approach to forming implants or engineered regenerative biostructures with three-dimensional printing is described in detail in co-owned U.S. application Ser. No. 10/122,129, filed Apr. 12, 2002, herein incorporated in its entirety by reference.

Another problem has been that solid metal implants have had a stiffness or Young's modulus that has been significantly greater than that of bone, which results in a situation called stress shielding. In such a situation, load is carried preferentially by the solid metal implant with the result that lightly-loaded bone adjacent to the implant resorbs due to the relatively light loading it experiences.

With the present invention including resorbable ceramic, in which eventually only a porous network of metal remains (surrounded by natural bone which has grown into it), the strength of the remaining metal would be less than that of a solid implant, resulting in less of a problem with stress shielding. The extent and dimensions of the metal-infiltrated region may be controlled by the controlled-infusion process described elsewhere herein or by other processes described herein, and may be chosen appropriately to reduce the problem of stress shielding.

This can be used for implants or more generally for medical devices which merely come into contact with the human body.

Nickel-titanium alloys have a substantial body of medical literature supporting their use inside the body, especially in connection with shape memory alloys. Also, zirconium has supporting literature from the nuclear fission industry supporting that when zirconium is added to otherwise corrosion-resistant alloys, the alloys continue to be corrosion-resistant.

The general intent of the present work has been to rely for melting point determination primarily on titanium, zirconium, niobium, nickel and stainless-steel-like alloys, i.e., the most biologically acceptable metals. The general intent of the present work has been to make little or no use of elements such as cobalt, chromium, molybdenum, vanadium, aluminum, manganese, silicon, phosphorus, sulfur, copper, tin etc., apart from their possible presence as unavoidable impurities, because there is some belief in the biomedical literature that a patient having a metal implanted device is better off if such elements are excluded.

However, it is possible there may be instances where it is acceptable to have the presence of these or other additional elements in some concentration, in addition to the main constituents of the alloys of the present invention as have already been described. For example, some of these elements have known uses as alpha stabilizers or beta stabilizers with titanium, or as precipitation hardeners. Accordingly, the patent application should also be understood to cover compositions which include described principal constituents having proportions, relative to each other, as described, but which additionally include small additional quantities of various other elements such as the just-listed elements.

All patents and applications cited above are incorporated by reference in their entirety. The above description of illustrated embodiments of the invention is not intended to be exhaustive or to limit the invention to the precise form disclosed. While specific embodiments of, and examples for, the invention are described herein for illustrative purposes, various equivalent modifications are possible within the scope of the invention, as those skilled in the relevant art will recognize.

Aspects of the invention can be modified, if necessary, to employ the process, apparatuses and concepts of the various patents and applications described above to provide yet further embodiments of the invention. These and other changes can be made to the invention in light of the above detailed description. From the foregoing it will be appreciated that, although specific embodiments of the invention have been described herein for purposes of illustration, various modifications may be made without deviating from the spirit and scope of the invention.

What is claimed is:

1. A composition for biomedical use which, other than unavoidable impurities, comprises nickel and titanium, wherein the relative proportions by weight of nickel and titanium range from 22 parts nickel 78 parts titanium, to 45 parts nickel 55 parts titanium.

2. The composition of claim 1 wherein the relative proportions by weight of nickel and titanium range from 24.5 parts nickel 75.5 parts titanium, to 41 parts nickel 59 parts titanium.

3. The composition of claim 2 wherein the relative proportions by weight of nickel and titanium range from 27 parts nickel 73 parts titanium, to 38 parts nickel 62 parts titanium.

4. The composition of claim 1 wherein the composition, other than unavoidable impurities, comprises only nickel and titanium.

5. The composition of claim 1 wherein the composition, other than unavoidable impurities, further comprises at least one other constituent.

6. The composition of claim 1 wherein the composition, other than unavoidable impurities, comprises only nickel and titanium and one or more elements known to be either alpha stabilizers or beta stabilizers for titanium.

7. The composition of claim 1 wherein the metal, other than unavoidable impurities, comprises only nickel and titanium and at least one element selected from the group consisting of aluminum, vanadium, molybdenum, zirconium, niobium, iron, tantalum, chromium, tungsten, hafnium, tin, oxygen and nitrogen.

8. A composition for biomedical use which, other than unavoidable impurities, comprises nickel and titanium, wherein the relative proportions by weight of nickel and titanium range from 63.5 parts nickel 36.5 parts titanium, to 67 parts nickel 33 parts titanium.

9. A composition for biomedical use which, other than unavoidable impurities, comprises nickel and zirconium, wherein the relative proportions by weight of nickel and zirconium range from 67 parts zirconium 33 parts nickel to 87 parts zirconium 13 parts nickel.

10. The composition of claim 9 wherein the relative proportions by weight of nickel and zirconium range from 70 parts zirconium 30 parts nickel, to 75 parts zirconium 25 parts nickel.

11. The composition of claim 9 wherein the relative proportions by weight of nickel and zirconium range from 77 parts zirconium 23 parts nickel, to 85 parts zirconium 15 parts nickel.

12. The composition of claim 9 further comprising at least one additional constituent.

13. The composition of claim 11 wherein the relative proportions by weight of nickel and zirconium range from 81 parts zirconium 19 parts nickel, to 84 parts zirconium 16 parts nickel.

14. A composition for biomedical use which, other than unavoidable impurities, comprises nickel and zirconium, wherein the relative proportions by weight of nickel and zirconium range from 41 parts zirconium 59 parts nickel to 54 parts zirconium 46 parts nickel.

15. The composition of claim 14 wherein the relative proportions by weight of nickel and zirconium range from 46 parts zirconium 54 parts nickel to 48 parts zirconium 52 parts nickel.

16. A composition for biomedical use which, other than unavoidable impurities, comprises nickel and zirconium, wherein the relative proportions by weight of nickel and zirconium range from 12.5 parts zirconium 87.5 parts nickel to 14 parts zirconium 86 parts nickel.

17. A composition for biomedical use which, other than unavoidable impurities, comprises nickel and niobium, wherein the relative proportions by weight of nickel and niobium range from 51 parts niobium 49 parts nickel to 53 parts zirconium 47 parts nickel.

18. A composition for biomedical use which, other than unavoidable impurities, comprises nickel in a weight fraction of from 60% to 87% and the balance being any one or more of titanium, zirconium and niobium in any combination or proportion.

19. The composition of claim 18 further comprising at least one additional constituent.

20. A composition for biomedical use which, other than unavoidable impurities, comprises a stainless-steel-like alloy and titanium, wherein the relative proportions by weight of stainless-steel-like alloy and titanium range from 12% titanium 88% stainless-steel-like alloy to 20% titanium 80% stainless-steel-like alloy, wherein the stainless-steel-like alloy is defined as any composition containing iron at greater than 50% by weight of the stainless-steel-like alloy, chromium ranging from 10% to 30% by weight of the stainless-steel-like alloy, and nickel ranging from 0 to 20% by weight of the stainless-steel-like alloy.

21. A composition for biomedical use which, other than unavoidable impurities, comprises a stainless-steel-like alloy and titanium, wherein the relative proportions by weight of stainless-steel-like alloy and titanium range from 60% titanium 40% stainless-steel-like alloy to 80% titanium 20% stainless-steel-like alloy, wherein the stainless-steel-like alloy is defined as any composition containing iron at greater than 50% by weight of the stainless-steel-like alloy, chromium ranging from 10% to 30% by weight of the stainless-steel-like alloy, and nickel ranging from 0 to 20% by weight of the stainless-steel-like alloy.

22. A composition for biomedical use which, other than unavoidable impurities, comprises a stainless-steel-like alloy and zirconium, wherein the relative proportions by weight of stainless-steel-like alloy and zirconium range from 15% zirconium 85% stainless-steel-like alloy to 25% zirconium 75% stainless-steel-like alloy, wherein the stainless-steel-like alloy is defined as any composition containing iron at greater than 50% by weight of the stainless-steel-like alloy, chromium ranging from 10% to 30% by weight of the stainless-steel-like alloy, and nickel ranging from 0 to 20% by weight of the stainless-steel-like alloy.

23. A composition for biomedical use which, other than unavoidable impurities, comprises a stainless-steel-like alloy and zirconium, wherein the relative proportions by weight of stainless-steel-like alloy and zirconium range from 60% zirconium 40% stainless-steel-like alloy to 90% zirconium 10% stainless-steel-like alloy, wherein the stainless-steel-like alloy is defined as any composition containing iron at greater than 50% by weight of the stainless-steel-like alloy, chromium ranging from 10% to 30% by weight of the stainless-steel-like alloy, and nickel ranging from 0 to 20% by weight of the stainless-steel-like alloy.

24. A composition for biomedical use which, other than unavoidable impurities, comprises a stainless-steel-like alloy and niobium, wherein the relative proportions by weight of stainless-steel-like alloy and niobium range from 15% niobium 85% stainless-steel-like alloy to 25% niobium 75% stainless-steel-like alloy, wherein the stainless-steel-like alloy is defined as any composition containing iron at greater than 50% by weight of the stainless-steel-like alloy, chromium ranging from 10% to 30% by weight of the stainless-steel-like alloy, and nickel ranging from 0 to 20% by weight of the stainless-steel-like alloy.

25. A composition for biomedical use which, other than unavoidable impurities, comprises a stainless-steel-like alloy and niobium, wherein the relative proportions by weight of stainless-steel-like alloy and niobium range from 50% niobium 50% stainless-steel-like alloy to 75% niobium 25% stainless-steel-like alloy, wherein the stainless-steel-like alloy is defined as any composition containing iron at greater than 50% by weight of the stainless-steel-like alloy, chromium ranging from 10% to 30% by weight of the stainless-steel-like alloy, and nickel ranging from 0 to 20% by weight of the stainless-steel-like alloy.

26. A composition for biomedical use which, other than unavoidable impurities, comprises titanium and zirconium, wherein the relative proportions by weight of titanium and zirconium range from approximately 30 percent zirconium to approximately 70% zirconium.

27. A composition for biomedical use which, other than unavoidable impurities, comprises chromium and niobium and nickel, wherein the relative proportions by weight are 20% chromium, 60% nickel, 20% niobium, plus or minus 5% in any of those concentrations.

28. A composition for biomedical use which, other than unavoidable impurities, comprises chromium and nickel and titanium, wherein the relative proportions by weight are chromium less than 30%, titanium greater than 10%, nickel greater than 10%.

29. A composition for biomedical use which, other than unavoidable impurities, comprises chromium and nickel and titanium, wherein the relative proportions by weight are chromium less than 10%, nickel between 30% and 40%, balance titanium.

30. An implant comprising a matrix containing a network of interconnected pores, wherein at least some of the pores are at least partially filled by a biocompatible metal.

31. The implant of claim 30 wherein the metal, other than unavoidable impurities, comprises only nickel and titanium.

32. The implant of claim 30 wherein the metal, other than unavoidable impurities, comprises only nickel and titanium and elements known to be either alpha stabilizers or beta stabilizers for titanium.

33. The implant of claim 30 wherein the metal, other than unavoidable impurities, comprises only nickel and titanium and at least one element selected from the group consisting of aluminum, vanadium, molybdenum, zirconium, niobium, iron, tantalum, chromium, tungsten, hafnium, tin, oxygen and nitrogen.

34. The implant of claim 30 wherein the metal, other than unavoidable impurities, comprises only nickel and zirconium.

35. The implant of claim 30 wherein the metal, other than unavoidable impurities, comprises only nickel and zirconium and at least one element selected from the group consisting of aluminum, vanadium, molybdenum, titanium, niobium, iron, tantalum, chromium, tungsten, hafnium, tin, oxygen and nitrogen.

36. The implant of claim 30 wherein the metal, other than unavoidable impurities, comprises only nickel and niobium.

37. The implant of claim 30 wherein the metal, other than unavoidable impurities, comprises only nickel and niobium and at least one element selected from the group consisting of aluminum, vanadium, molybdenum, zirconium, titanium, iron, tantalum, chromium, tungsten, hafnium, tin, oxygen and nitrogen.

38. The implant of claim 30 wherein the metal has a melting point of less than approximately 1200° C.

39. The implant of claim 30 wherein the metal has a melting point less than approximately 1100° C.

40. The implant of claim 30 wherein the metal has a melting point less than approximately 1000° C.

41. An implant comprising a ceramic matrix containing a network of interconnected pores, wherein at least some of the pores are at least partially filled by a biocompatible metal.

42. The implant of claim 41 wherein the ceramic matrix also contains macroscopic channels, sprues, or runners, at least some of the channels sprues or runners being at least partially filled by the biocompatible metal.

43. The implant of claim 41 wherein the ceramic is a member of the calcium phosphate family.

44. The implant of claim 41 wherein the ceramic is nonresorbable.

45. The implant of claim 41 wherein the ceramic is resorbable.

46. The implant of claim 41 wherein the ceramic is beta tricalcium phosphate.

47. The implant of claim 41 wherein the metal has a melting point less than approximately 1200° C.

48. The implant of claim 41 wherein the metal has a melting point less than approximately 1100° C.

49. The implant of claim 41 wherein the metal has a melting point less than approximately 1000° C.

50. An article for biomedical use, comprising the composition of claim 29.

51. A bone substitute comprising the composition of claim 28.

52. A bone substitute, wherein the bone substitute comprises a first network of a first material interpenetrating with a second network of the composition of claim 27.

53. The bone substitute of claim 51, wherein the first network comprises ceramic.

54. The bone substitute of claim 51, wherein the first network comprises a resorbable ceramic.

55. The bone substitute of claim 54, wherein the resorbable ceramic comprises beta tricalcium phosphate.

56. The bone substitute of claim 51, wherein the metal has a melting point of lower than 1200° C.

57. A bone substitute comprising metal having a composition of 26 from 22 parts nickel 78 parts titanium to 45 parts nickel 55 parts titanium, or from 63.5 parts nickel 36.5 parts titanium to 67 parts nickel 33 parts titanium.

58. The bone substitute of claim 57, wherein the metal has a melting point of lower than 1200° C.

59. A biomedical article comprising a matrix containing a network of interconnected pores, wherein at least some of the pores are at least partially filled by a biocompatible metal.

60. A method of manufacturing a biomedical article, comprising:
manufacturing a matrix having pores;
infusing into the pores an infiltrant which is a metal composition having a melting point less than 1200° C.; and
allowing the infiltrant to harden.

61. The method of claim 60, wherein infusing the infiltrant comprises infusing a composition in the range from 22 parts nickel 78 parts titanium to 45 parts nickel 55 parts titanium, or from 63.5 parts nickel 36.5 parts titanium to 67 parts nickel 33 parts titanium.

62. The method of claim 60 wherein manufacturing the matrix comprises manufacturing a matrix comprising a ceramic material.

63. The method of claim 60 wherein manufacturing the matrix comprises manufacturing a matrix comprising beta tricalcium phosphate.

64. The method of claim 60 wherein manufacturing the matrix comprises manufacturing a matrix comprising a metal.

65. The method of claim 60 wherein the matrix comprises a metal that is a constituent of the infiltrant.

66. The method of claim 60 wherein the matrix comprises a metal which is a constituent of the infiltrant and wherein the allowing the infiltrant to harden comprises holding the article for a period of time at a temperature which is greater than a melting temperature of the infiltrant but less than a temperature at which the matrix and the infiltrant would solidify if the constituents of the matrix and the infiltrant were evenly distributed.
